(19) United States
(12) Patent Application Publication
(10) Pub. No.: US 2023/0332131 A1
(43) Pub. Date: Oct. 19, 2023

(54) PRODUCTION OF ANTIGEN-SPECIFIC T-CELLS

(71) Applicants: NEXIMMUNE, INC., Gaithersburg, MD (US); THE JOHNS HOPKINS UNIVERSITY, Baltimore, MD (US)

(72) Inventors: Mathias OELKE, Gaithersburg, MD (US); Jose Luis SANTOS, Gaitherburg, MD (US); Sojung KIM, Gaitherberg, MD (US); Jonathan SCHNECK, Baltimore, MD (US); Alyssa KOSMIDES, Baltimore, MD (US)

(21) Appl. No.: 18/111,975

(22) Filed: Feb. 21, 2023

Related U.S. Application Data
(63) Continuation of application No. 16/084,121, filed on Sep. 11, 2018, now abandoned.
(60) Provisional application No. 62/309,234, filed on Mar. 16, 2016.

(51) Int. Cl. C12N 13/00; A61K 39/00; C12N 5/0783; A61K 35/17; C12N 15/10; C07K 16/00; C12Q 1/6881

(52) U.S. Cl. CPC C12N 13/00; A61K 35/17; A61K 39/001114; C07K 16/00; C12N 5/0636; C12N 15/1079; C12Q 1/6881; C07K 2317/24; C07K 2319/03; C07K 2319/33

(57) ABSTRACT

The invention in various aspects provides for magnetic enrichment and/or expansion of antigen-specific T cells, allowing for identification and characterization of antigen-specific T cells and their T cell receptors (TCRs) for therapeutic and/or diagnostic purposes, as well as providing for production of antigen-specific engineered T cells for therapy. Incubation of paramagnetic nano-aAPCs in the presence of a magnetic field, either during enrichment and/or expansion steps, activates T cells through magnetic clustering of paramagnetic particles on the T cell surface.

### STATEMENT OF RIGHTS TO INVENTIONS MADE UNDER FEDERALLY SPONSORED RESEARCH

This invention was made with government support under grant number CA108835, awarded by the National Institutes of Health (NIH). The government has certain rights in the invention.

### BACKGROUND

Expansion of antigen-specific T cells is complicated by the rarity of antigen-specific naive precursors, which can be as few as one per million. To generate the large numbers of tumor-specific T cells required for adoptive therapy (for example), lymphocytes are conventionally stimulated with antigen over many weeks, often followed by T cell selection and sub-cloning in a labor intensive process. Further, various processes currently in use for expanding lymphocytes, such as anti-CD3/anti-CD28 beads, have a tendency to produce T cells that exhibit somewhat of an exhausted phenotype. See, Sachamitr P. et al., Induced pluripotent stem cells: challenges and opportunities for cancer immunotherapy, Front Immunol. 2014 Apr 17;5:176.

There is a need for technologies that can quickly generate large numbers and/or high frequencies of antigen-specific T cells, including T cells that do not exhibit an exhausted phenotype, for both therapeutic and diagnostic purposes.

### SUMMARY OF THE INVENTION

The invention in various aspects provides for magnetic enrichment and/or expansion of antigen-specific T cells, allowing for identification and characterization of antigen-specific T cells and their T cell receptors (TCRs) for therapeutic and/or diagnostic purposes, as well as providing for production of antigen-specific engineered T cells for therapy. Incubation of paramagnetic nano-aAPCs in the presence of a magnetic field, either during enrichment and/or expansion steps, activates T cells through magnetic clustering of paramagnetic particles on the T cell surface.

In various aspects, the invention provides methods for expanding antigen-specific T cell populations for adoptive immunotherapy, including engineered T cells that express a heterologous T cell receptor or a chimeric antigen receptor (CAR). T cells expanded in accordance with embodiments of the invention display a polyfunctional phenotype (Tcm, Tem), as opposed to T cells expanded non-specifically with anti-CD3/anti-CD28, which are closer to an exhausted phenotype.

In some embodiments, the invention provides artificial antigen-presenting cells especially configured for magnetic enrichment and expansion of antigen-specific T cells, including the separation of antigen presenting complexes (signal 1) and lymphocyte co-stimulatory signals (signal 2) (e.g., anti-CD28) on separate beads to allow additional levels of control and variation of the process.

In still other aspects, the invention provides methods for screening large numbers of candidate antigens for reactivity specificity in a T cell population. The method employs sequential enrichment of antigen-specific T cells with a magnetic column and paramagnetic aAPCs, with the negative fraction used for subsequent enrichment steps. Several candidate antigens can be batched in each enrichment step, through presentation by a cocktail of aAPCs presenting different peptide antigens. Since each step of sequential enrichment can screen a number of candidate antigenic peptides, the method easily allows for at least 75 antigens to be tested, without diluting the frequency of antigen-specific T cell precursors in the original sample.

In exemplary embodiments, the invention provides methods of treating patients having a hematological malignancy, such as acute myelogenous leukemia (AML) or myelodysplastic syndrome. In some embodiments, the patient has relapsed after allogeneic stem cell transplantation. Using a source of T cells from an HLA matched donor, antigen-specific T cells are magnetically enriched and activated using a magnetic column with paramagnetic nano-aAPC(s) presenting at least 2 or 3 tumor associated peptide antigens. Peptide antigens are passively loaded onto prepared nano-aAPCs, with ligands chemically conjugated to the particles through free cysteines that have been engineered into the proteins near the C-terminal end of the Fc portions of immunoglobulin sequences. For example, aAPCs may comprise signal 1 and signal 2 on the same or different populations of nano-particles.

In some embodiments, the magnetic activation takes place for at least 5 minutes, such as from 5 minutes to 5 hours or from 5 minutes to 2 hours, followed by expansion in culture for at least 5 days, and up to 3 weeks in some embodiments. Resulting CD8+ T cells may be phenotypically characterized to confirm that they are of central memory or effector memory phenotype and poly functional. Expanded T cells can be administered to the patient to establish an anti-tumor response.

Other aspects and embodiments of the invention will be apparent from the following detailed description.

### DESCRIPTION OF THE FIGURES

FIG. 1 shows signal 1 and signal 2 in the context of T cell activation (left panel), and the construction of artificial antigen presenting cells on paramagnetic particles (right panel). Only cognate T cells are activated by aAPCs.

FIG. 2 illustrates different co-stimulatory signals (signal 2) that may be presented on nanoparticles in accordance with embodiments of the invention, and illustrates the control of signal 2 achieved by placing signal 2 on separate particles.

FIG. 3 demonstrates clustering of paramagnetic particles with T cell co-receptor (CD3ε) on the surface of T cells in the presence of a magnetic field.

FIG. 4 shows that the presence of a magnetic field enhances proliferation of T cells with the paramagnetic aAPCs, and that this enhancement is dependent on the amount of signal 2 present on a separate nanoparticle.

FIG. 5A and FIG. 5B show that signal 1 and signal 2 can support T cell expansion even when present on separate nanoparticles (FIG. 5A, left panel), and that the resultant CD8 T cells are equivalent to those activated by aAPC presenting both signals (FIG. 5A, right panel). FIG. 5B shows cytokine secretion profiles (number of cytokines or effector molecules secreted) of T cells activated with aAPC presenting both signals, as compared to having signals presented on separate particles.

FIG. 6A and FIG. 6B illustrate the clustering of paramagnetic beads containing separate signal 1 and signal 2 in the presence of a magnetic field, as compared to polystyrene particles that do not cluster (FIG. 6A), and the increased expansion observed with the magnetic expansion system (FIG. 6B).

FIG. 7 shows that optimal T cell expansion is seen where signal 1 and 2 are clustered sufficiently close. As particle size increases, the efficacy of the S1+S2 approach decreases (right panel). In contrast, nanoparticles containing both signals show the opposite effect (left panel).

FIG. 8 shows that the types of co-stimulation can be varied to customize the activation profile.

FIG. 9 shows the gating scheme used to purify cells prior to sequencing their clonotypic T cell receptor. Initially naïve T cells were taken and stimulated with nano-aAPC using the E+E system. At day 7, cells were harvested and analyzed by flow cytometry. The left panel shows the total number of events seen in the culture and gated on the lymphocyte population. In the middle panel, live/dead cells were stained and gated exclusively on the live cells, and in the right panel the MHC_Ig dimer loaded with the trp-2 peptide was used to stain, and only the positive cells were sorted (approximately 18.3%). These cells were then sent for TCR sequencing and results are shown in FIG. 10.

FIG. 10 shows the number of productive and non-productive clones, based on TCR sequencing analysis.

FIG. 11A and FIG. 11B compare the frequencies of top clones (identified as >0.1% frequency and > 100 reads in Carreno et al, Science 15;348(6236):803-8 (2015)) (FIG. 11A), as compared to frequencies of productive clones after magnetic enrichment and expansion (FIG. 11B).

FIG. 12 shows frequencies of T cell clonotypes based on percent of total reads.

FIG. 13 is a 3D histogram of V and J pairing frequency for all clones.

FIG. 14 is a 3D histogram of V and J pairing frequency for top 10 clones, based on total reads.

FIG. 15 shows generation of functionally active human neo-antigen-specific CD-8+ T cells from a healthy donor. Three neo-epitopes from MCF-7 breast cancer were tested simultaneously using the magnetic enrichment and expansion process.

FIG. 16 shows that passive loading of peptide to nanoparticles having site-directed MHC conjugation provided an increased expansion after 1 week.

### DETAILED DESCRIPTION

The invention in various aspects provides for magnetic enrichment and/or magnetic expansion of antigen-specific T cells, allowing for identification and characterization of antigen-specific T cells and their T cell receptors (TCRs) for therapeutic and/or diagnostic purposes, as well as providing for production of antigen-specific engineered T cells for therapy. Magnetic enrichment refers to the use of paramagnetic nanoparticles having on their surface an MHC-peptide antigen presenting complex, such that antigen specific T cells can be separated from a T cell population by a magnetic column, while other cells (including non-cognate T cells) pass through. Expansion of enriched T cells can take place in the presence or absence of a magnetic field. Magnetic enhanced expansion refers to the expansion and/or activation of T cells using paramagnetic nanoparticles having on their surface an MHC-peptide antigen presenting complex and one or more lymphocyte co-stimulatory ligands (which may be on the same or different particles), such that the presence of a magnetic field induces magnetic clustering of the nanoparticles and TCRs, thereby driving activation and subsequent expansion of the antigen-specific T cell fraction. Magnetic clustering of nanoscale artificial antigen presenting cells to drive T cell expansion is disclosed in US 2016/0051698, which is hereby incorporated by reference. In various embodiments, the process of enrichment and expansion includes magnetic activation, in which paramagnetic nano-aAPCs harboring signal 1 and signal 2 (either on the same of different populations of nanoparticles) are incubated in the presence of a magnetic field. The incubation in the presence of a magnetic field generally takes place for at least 5 minutes, or at least 10 minutes, or at least 15 minutes, or at least 30 minutes, or at least one hour, or at least 2 hours. For example, the incubation in the presence of a magnetic field may take place for 5 minutes to about 2 hours or from about 10 minutes to about 1 hour.

In various aspects, the invention provides methods for expanding antigen-specific T cell populations for adoptive immunotherapy, including engineered T cells that express a heterologous T cell receptor or chimeric antigen receptor (CAR). Adoptive immunotherapy involves the activation and expansion of immune cells ex vivo, with the resulting cells transferred to the patient to treat disease, such as cancer. Induction of antigen-specific cytotoxic (CD8+) lymphocyte (CTL) responses, for example, through adoptive transfer could be an attractive therapy, if sufficient numbers and frequency of activated and antigen-specific CTL can be generated in a relatively short time, including from rare precursor cells. This approach in some embodiments could even generate long-term memory that prevents recurrence of disease. In addition to cancer immunotherapy, and immunotherapies involving CTLs, the invention finds use with other immune cells, including CD4+ T cells and regulatory T cells, and thus is broadly applicable to immunotherapy for infectious disease and auto-immune disease, among others. Further, T cells expanded in accordance with embodiments of the invention display a polyfunctional phenotype (Tcm, Tem), as opposed to T cells expanded non-specifically with anti-CD3/anti-CD28, which are closer to an exhausted phenotype.

In some embodiments, T cells having a central memory (Tcm) or effector memory (Tem) phenotype are produced according to the following disclosure, and then a chimeric antigen receptor or heterologous TCR is introduced into the cell to produce a CAR-T cell for adoptive therapy. Such cells can be activated and expanded in vivo using the processes described herein.

In still other embodiments, the nanoparticle comprises ligands that engage with a CAR-T receptor, such as CD19, as signal 1. Nanoparticles according to these embodiments allow for magnetic activation and subsequent expansion of CAR-T cells.

For example, in various embodiments, CD8+ lymphocytes expanded in accordance with embodiments of the invention comprise the following phenotypes: low PD-1 expression; central memory phenotype (CD3+, CD44+, CD62L+); and effector memory phenotype (CD3+, CD44+, CD62L-). In some embodiments, CD8+ lymphocytes enriched and expanded in accordance with embodiments of the invention produce proinflammatory markers such IFNγ, TNFα, IL-2, MIP-1β, GrzB, and/or perforin when stimulated with aAPCs loaded with cognate antigen.

In some aspects, the invention provides a method for rapidly generating large numbers of antigen-specific T cells, which can be phenotypically and/or genotypically characterized to identify productive and effective antigen-specific TCRs. For example, in this aspect, the invention provides a method for identifying an antigen-specific T cell Receptor (TCR). The method comprises magnetically enriching and/or magnetically expanding a heterogeneous T cell population with paramagnetic nanoparticles having an MHC-peptide antigen presenting complex on the surface, as described in more detail herein. The expanded T cells are then sorted (e.g., by flow cytometry) with the MHC-peptide ligand, to obtain a T cell population that is highly enriched for antigen-specific TCRs. The TCR repertoire can then be sequenced and/or profiled. Together with functional characterization of the expanded T cells, TCRs with defined affinities can be identified in a short time. Such TCRs find use for heterologous expression to generate engineered T cells for adoptive therapy.

The invention is this aspect allows for sufficient numbers of T cells to be generated for sequencing in only a few days. For example, in some embodiments, magnetically enriched cells are expanded in culture for about 2 days to up to 9 weeks, or in some embodiments, from 5 days to about 2 weeks (e.g., about 1 week). DNA sequencing can be conducted using any known process, including pyrosequencing, next generation sequencing (NGS; DNA or RNA sequencing) or sequencing-by-synthesis. Sequencing generally includes the TCR alpha and/or beta chains, including complementarity-determining regions of the TCR, e.g., CDR3 of the beta receptor chain, formed by V, D and J gene regions.

In another aspect, the invention provides a method for screening a T cell population for reactivity to a library of candidate antigenic peptides. In various embodiments, the method comprises magnetically enriching and magnetically expanding antigen-specific T cells in the population with a cocktail of paramagnetic nanoparticles, each having MHC-peptide antigen presenting complexes on the surface thereof that presents a candidate antigenic peptide. The method further comprises phenotypically evaluating the enriched and expanded T cells, e.g., for their reactivity with the candidate peptides.

In some embodiments, sequential magnetic enrichment is performed with the flow-through fraction from the initial magnetic enrichment step, each sequential enrichment employing a different antigenic peptide of interest, or a different set of antigenic peptides. For example, in some embodiments at least 6, or at least 10, or at least 20 sequential magnetic enrichments are performed. Since each step of sequential enrichment can screen from 5 to about 20 candidate antigenic peptides, the method allows for 30 to 400 antigens to be tested. In various embodiments, at least 50 antigens are tested, or at least 75 antigens are tested, or at least 100 antigens are tested, or at least 150 antigens to be tested, or at least 200 antigens are tested, or at least 300 antigens are tested, without diluting the frequency of antigen-specific T cell precursors in the original sample.

In other aspects, the invention provides methods for expansion of T cells comprising a heterologous or engineered T cell receptor (TCR). The method comprises magnetically enriching and magnetically expanding a T cell population that comprises T cells expressing a heterologous or engineered T cell receptor (TCR). Enrichment and expansion is conducted with paramagnetic nanoparticles having an MHC-peptide antigen presenting complex recognized by the heterologous or engineered T cell receptor (TCR) on the surface of the particles. In some embodiments, starting with antigen-specific frequencies of from about 10% to about 40% (e.g., at least about 20%), the method produces high frequency and numbers of antigen-specific T cells within about 10 to 14 days.

In other aspects, the invention provides a method for preparing an antigen-specific T-cell population by magnetic enrichment and expansion, wherein the MHC-peptide complex is prepared by passive loading of MHC-conjugated nanoparticles. Passive loading of nanoparticles is contrasted with refolding of the MHC in the presence of peptide, followed by conjugation or attachment of the antigen presenting complex to the surface of particles. By preparing batches of particles that are uncommitted to particular antigenic peptides, the work flow and cost of the process is greatly improved. As disclosed in U.S. Patent 6,734,013, which is hereby incorporated by reference in its entirety, active loading of peptide antigen to MHC-Ig with alkaline stripping, rapid neutralization, and refolding in the presence of peptide produced ligands that were 10 to 100-fold more potent for T cell staining than corresponding passively-loaded MHC-Ig. However, embodiments of the present invention provide for robust enrichment and expansion of antigen-specific T cells with superior functionality using even passively loaded HLA-Ig ligands. For example, in some embodiments, the MHC-conjugated nanoparticles are passively loaded for at least about 2 days by incubation with excess peptide antigen.

While magnetic enrichment and expansion has been described with aAPCs that contain both signal 1 (MHC-peptide complex) and signal 2 (e.g., anti-CD28), in the various aspects of the invention, the aAPCs in some embodiments only contain signal 1. A second nanoparticle having a lymphocyte co-stimulatory ligand conjugated to its surface is added during the enrichment step or during expansion of recovered T cells. By providing the “signal 2” (e.g., lymphocyte costimulatory ligand) on a separate particle, the timing and type of stimulus can be controlled. The second nanoparticle may also be paramagnetic, allowing the second particle to magnetically cluster with first nanoparticles presenting the MHC-peptide antigen presenting complex. In these embodiments, the nanoparticles are preferably kept small, such as less than about 200 nm, less than about 100 nm, or less than about 50 nm. Thus, in some embodiments, the second nanoparticle is paramagnetic, and the second nanoparticle is added during the expansion of T cells recovered during the enrichment step(s).

In some embodiments, the second nanoparticle is not paramagnetic, and is added during the magnetic enrichment of antigen-specific T cells. Because the signal 2 nanoparticle will not be magnetically bound by the column, the signal 2 nanoparticles will not lead to magnetic capture of non-specific T cells. In some embodiments, the non-paramagnetic nanoparticle approach is used for sequential enrichment, to avoid loss or unwanted retention of non-cognate T cells in each enrichment step. The second nanoparticle can be any non-paramagnetic material, including any of the known polymeric materials, including polystyrene or latex particles, or particles that comprise PLGA, PLGA-PEG, PLA, or PLA-PEG.

In still other aspects, the invention provides a method for generating a T cell expressing a chimeric antigen receptor (CAR), the method comprising magnetically enriching and expanding a T cell population with paramagnetic nanoparticles having an MHC-peptide antigen presenting complex on the surface thereof, to thereby prepare an enriched and expanded antigen-specific T cell population; and transforming the T cell population with a chimeric antigen receptor (CAR).

In various embodiments, the patient is a cancer patient, and the expanded CAR-T cells may be adoptively transferred to the patient, optionally with reactivation by administration of biocompatible aAPCs. In some embodiments, the method comprises boosting with a pharmaceutical composition comprising an artificial antigen-presenting cell (aAPC) presenting the MHC-peptide antigen-presenting complex and a lymphocyte co-stimulatory ligand, to thereby expand and reactivate the CAR-T cells in vivo. Suitable aAPCs for therapeutic use are described in WO 2016/105542, which is hereby incorporated by reference in its entirety.

In a related embodiment, the invention provides a method for expanding a T cell expressing a CAR, to enhance the production process. For example, the method may comprise providing the T cell population expressing a CAR as described above, and magnetically enriching and/or expanding the T cell population in the presence of paramagnetic nanoparticles having an MHC-peptide antigen presenting complex on the surface thereof.

Exemplary CARs include fusions of single-chain variable fragments (scFv) derived from monoclonal antibodies, fused to CD3-zeta transmembrane and endodomain, or other TCR signaling domain. The CAR may target malignant B cells by targeting CD19, for example.

In the various aspects, the present invention employs artificial Antigen Presenting Cells (aAPCs), which capture and deliver stimulatory signals to immune effector cells, such as antigen-specific T lymphocytes, such as CTLs. Signals present on the aAPCs that support T cell activation include Signal 1, antigenic peptide presented in the context of Major Histocompatibility Complex (MHC), class I or class II, and which bind antigen-specific T-cell Receptors (TCR); and Signal 2, one or more co-stimulatory ligands that modulate T cell response. As described herein, Signal 1 and Signal 2 can be supplied on separate particles, and the selection of the particle material for Signal 2 (e.g., paramagnetic or non-paramagnetic), can provide additional functionalities to the methods. Signal 1 and signal 2 ligands can be chemically conjugated to nanoparticles in a site directed fashion, such that ligands maintain a functional orientation on the particles.

In some embodiments of this system, Signal 1 is conferred by a monomeric, dimeric or multimeric MHC construct. A dimeric construct is created in some embodiments by fusion to a variable region or CH1 or CH2 region of an immunoglobulin heavy chain sequence. The MHC complex is loaded with one or more antigenic peptides. Signal 2 is either B7.1 (the natural ligand for the T cell receptor CD28) or an activating antibody against CD28. The Signal 1 and Signal 2 ligands may include variations in glycosyl groups or modification of free cysteine sulfhydryl groups.

In some aspects, the invention provides a method for preparing an antigen-specific T-cell population for adoptive transfer. In these aspects, T-cells are from a patient or a suitable donor. The aAPCs may present antigens that are common for the disease of interest (e.g., tumor-type), or may present one or more antigens selected on a personalized basis. The expansion step can proceed for about 3 days to about 2 weeks in some embodiments, or about 5 days to about 10 days (e.g., about 1 week). The enrichment and expansion process may then be repeated one or more times, for optimal expansion (and further purity) of antigen-specific cells. For subsequent rounds of enrichment and expansion, additional aAPCs may be added to the T cells to support expansion of the larger antigen-specific T cell population in the sample. In certain embodiments, the final round (e.g., round 2, 3, 4, or 5) of expansion occurs in vivo, where biocompatible nanoAPCs are added to the expanded T cell population, and then infused into the patient.

In certain embodiments, the method provides for about 1000-10,000 fold expansion (or more) of antigen-specific T cells, with more than about 10⁸ antigen-specific T cells being generated in the span of, for example, less than about one month, or less than about three weeks, or less than about two weeks, or in about one week. The resulting cells can be administered to the patient to treat disease. The aAPC may be administered to the patient along with the resulting antigen-specific T cell preparation in some embodiments.

When selecting T cell antigens on a personalized basis, a library of aAPCs each presenting a candidate antigenic peptide is screened with T cells from a subject or patient, and the response of the T cells to each aAPC-peptide is determined or quantified. T cell response can be quantified molecularly in some embodiments, for example, by quantifying cytokine expression or expression of other surrogate marker of T cell activation (e.g., by immunochemistry or amplification of expressed genes such as by RT-PCR). In some embodiments, the quantifying step is performed between about 15 hours and 48 hours in culture. In other embodiments, T cell response is determined by detecting intracellular signaling (e.g., Ca2+ signaling, or other signaling that occurs early during T cell activation), and thus can be quantified within about 15 minutes to about 5 hours (e.g., within about 15 minutes to about 2 hours) of culture with the nano-aAPCs. Peptides showing the most robust responses are selected for immunotherapy, including in some embodiments the adoptive immunotherapy approach described herein. In some embodiments, and particularly for cancer immunotherapy, a patient’s tumor is genetically analyzed (e.g., using next generation sequencing), and tumor antigens are predicted from the patient’s unique tumor mutation signature (e.g., comprising unique mutations in the DNA of the patient’s tumor that do not occur in non-tumor cells). These predicted antigens (“neoantigens”) are synthesized and screened against the patient’s T cells using the aAPC platform described herein. Once reactive antigens are identified/confirmed, aAPCs can be prepared for the enrichment and expansion protocol described herein, or the aAPCs can be directly administered to the patient in some embodiments.

In some aspects, a subject or patient’s T cells are screened against an array or library of paramagnetic nano-aAPCs (as described herein), where each paramagnetic nano-aAPC presents a peptide antigen. T cell responses to each are determined or quantified, providing useful information concerning the patient’s T cell repertoire, and hence the condition of the subject or patient. For example, the number and identity of T cell anti-tumor responses against mutated proteins, overexpressed proteins, and/or other tumor-associated antigens can be used as a biomarker to stratify risk, and in some embodiments can involve a computer-implemented classifier algorithm to classify the response profile for drug resistance or drug sensitivity, or stratify the response profile as a candidate for immunotherapy (e.g., checkpoint inhibitor therapy or adoptive T cell transfer therapy). For example, the number or intensity of such T cell responses may be inversely proportionate to a high risk of disease progression, and/or may directly relate to the patient’s likely response to immunotherapy, which may include one or more of checkpoint inhibitor therapy, adoptive T cell transfer, or other immunotherapy for cancer.

In still other aspects and embodiments, the patient’s T cells are screened against an array or library of paramagnetic nano-APCs, each presenting a candidate peptide antigen. For example, the array or library may present tumor-associated antigens, or may present auto-antigens, or may present T cell antigens relating to various infectious diseases. By incubating the array or library with the patient’s T cells, and in the presence of a magnetic field to encourage T cell receptor clustering, the presence of T cells responses, and/or the number or intensity of these T cells responses, can be rapidly determined. This information is useful for diagnosing, for example, a sub-clinical tumor, an autoimmune or immune disease, or infectious disease, and can provide an initial understanding of the disease biology, including, potential pathogenic or therapeutic T cells, T cell antigens, and an understanding of the T cell receptors of interest, which represent drug or immunotherapy targets.

The present invention provides for immunotherapy for cancer and other diseases in which detection, enrichment and/or expansion of antigen-specific immune cells ex vivo is therapeutically or diagnostically desirable. The invention is generally applicable for detection, enrichment and/or expansion of antigen-specific T cells, including cytotoxic T lymphocytes (CTLs), helper T cells, and regulatory T cells, as well as NKT cells or even B cells where the corresponding ligand were presented on the surface of the aAPC.

In some embodiments, the patient is a cancer patient. The enrichment and expansion of antigen-specific CTLs ex vivo for adoptive transfer to the patient provides for a robust anti-tumor immune response. Cancers that can be treated or evaluated according to the methods include cancers that historically illicit poor immune responses or have a high rate of recurrence. Exemplary cancers include various types of solid tumors, including carcinomas, sarcomas, and lymphomas. In various embodiments the cancer is melanoma (including metastatic melanoma), colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, neuroblastoma, and glioma.

In some embodiments, the cancer is a hematological malignancy, including leukemia, lymphoma, or myeloma. For example, the hematological malignancy may be acute myeloid leukemia, chronic myelogenous leukemia, childhood acute leukemia, non-Hodgkin’s lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia.

In various embodiments, the cancer is stage I, stage II, stage III, or stage IV. In some embodiments, the cancer is metastatic and/or recurrent. In some embodiments, the cancer is preclinical, and is detected in the screening system described herein (e.g., colon cancer, pancreatic cancer, or other cancer that is difficult to detect early).

In some embodiments, the patient has an infectious disease. The infectious disease may be one in which enrichment and expansion of antigen-specific immune cells (such as CD8+ or CD4+ T cells) ex vivo for adoptive transfer to the patient could enhance or provide for a productive/protective immune response. Infectious diseases that can be treated include those caused by bacteria, viruses, prions, fungi, parasites, helminths, etc. Such diseases include AIDS, hepatitis B/C, CMV infection, and post-transplant lymphoproliferative disorder (PTLD). CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals. A useful alternative to these treatments is a prophylactic immunotherapeutic regimen involving the generation of virus-specific CTL derived from the patient or from an appropriate donor before initiation of the transplant procedure. PTLD occurs in a significant fraction of transplant patients and results from Epstein-Barr virus (EBV) infection. EBV infection is believed to be present in approximately 90% of the adult population in the United States. Active viral replication and infection is kept in check by the immune system, but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers. For infectious diseases involving biofilms, or a matrix supporting bacterial growth in a non-planktonic state, CD8+ T cells can be important for resolution. Antigen-specific responses that recruit activated CD8+ T cells which infiltrate the biofilm matrix could prove effective for the elimination of antibiotic resistant microbial infection.

In some embodiments, the patient has an autoimmune disease, in which enrichment and expansion of regulatory T cells (e.g., CD4+, CD25+, Foxp3+) ex vivo for adoptive transfer to the patient could dampen the deleterious immune response. Autoimmune diseases that can be treated include systemic lupus erythematosus, rheumatoid arthritis, type I diabetes, multiple sclerosis, Crohn’s disease, ulcerative colitis, psoriasis, myasthenia gravis, Goodpasture’s syndrome, Graves’ disease, pemphigus vulgaris, Addison’s disease, dermatitis herpetiformis, celiac disease, and Hashimoto’s thyroiditis. In some embodiments, the patient is suspected of having an autoimmune disease or immune condition (such as those described in the preceding sentence), and the evaluation of T cell responses against a library of paramagnetic nano-aAPCs as described herein, is useful for identifying or confirming the immune condition.

Thus, in various embodiments the invention involves enrichment and expansion of antigen-specific T cells, such as cytotoxic T lymphocytes (CTLs), helper T cells, or regulatory T cells. In some embodiments, the invention involves enrichment and expansion of antigen-specific CTLs. Precursor T cells can be obtained from the patient or from a suitable HLA-matched donor. Precursor T cells can be obtained from a number of sources, including peripheral blood mononuclear cells (PBMC), bone marrow, lymph node tissue, spleen tissue, and tumors. In some embodiments, the sample is a PBMC sample from the patient. In some embodiments, the PBMC sample is used to isolate the T cell population of interest, such as CD8+, CD4+ or regulatory T cells. In some embodiments, precursor T cells are obtained from a unit of blood collected from a subject using any number of techniques known to the skilled artisan, such as Ficoll separation. For example, precursor T cells from the circulating blood of an individual can be obtained by apheresis or leukapheresis. The apheresis product typically contains lymphocytes, including T cells and precursor T cells, monocytes, granulocytes, B cells, other nucleated white blood cells, red blood cells, and platelets. Leukapheresis is a laboratory procedure in which white blood cells are separated from a sample of blood.

Cells collected by apheresis can be washed to remove the plasma fraction and to place the cells in an appropriate buffer or media for subsequent processing steps. Washing steps can be accomplished by methods known to those in the art, such as by using a semi-automated “flow-through” centrifuge (for example, the Cobe 2991 cell processor) according to the manufacturer’s instructions. After washing, the cells may be resuspended in a variety of biocompatible buffers, such as, for example, Ca-free, Mg-free PBS. Alternatively, the undesirable components of the apheresis sample can be removed and the cells directly re-suspended in a culture medium.

If desired, precursor T cells can be isolated from peripheral blood lymphocytes by lysing the red blood cells and depleting the monocytes, for example, by centrifugation through a PERCOLL™ gradient.

If desired, subpopulations of T cells can be separated from other cells that may be present. For example, specific subpopulations of T cells, such as CD28+, CD4+, CD8+, CD45RA+, and CD45RO+T cells, can be further isolated by positive or negative selection techniques. Other enrichment techniques include cell sorting and/or selection via negative magnetic immunoadherence or flow cytometry, e.g., using a cocktail of monoclonal antibodies directed to cell surface markers present on the cells negatively selected.

In certain embodiments, leukocytes are collected by leukapheresis, and are subsequently enriched for CD8+ T cells using known processes, such as magnetic enrichment columns that are commercially available. The CD8-enriched cells are then further enriched for antigen-specific T cells using magnetic enrichment with the aAPC reagent. In various embodiments, at least about 10⁵, or at least about 10⁶, or at least about 10⁷ CD8-enriched cells are isolated for antigen-specific T cell enrichment.

In various embodiments, the sample comprising the immune cells (e.g., CD8+ T cells) is contacted with an artificial Antigen Presenting Cell (aAPC) having magnetic properties. Paramagnetic materials have a small, positive susceptibility to magnetic fields. These materials are attracted by a magnetic field and the material does not retain the magnetic properties when the external field is removed. Exemplary paramagnetic materials include, without limitation, magnesium, molybdenum, lithium, tantalum, and iron oxide. Paramagnetic beads suitable for magnetic enrichment are commercially available (DYNABEADSTM, MACS MICROBEADSTM, Miltenyi Biotec). In some embodiments, the aAPC particle is an iron dextran bead (e.g., dextran-coated iron-oxide bead).

Antigen presenting complexes comprise an antigen binding cleft, which harbors an antigen for presentation to a T cell or T cell precursor. Antigen presenting complexes can be, for example, MHC class I or class II molecules, and can be linked or tethered to provide dimeric or multimeric MHC. In some embodiments, the MHC are monomeric, but their close association on the nano-particle is sufficient for avidity and activation. In some embodiments, the MHC are dimeric. Dimeric MHC class I constructs can be constructed by fusion to immunoglobulin heavy chain sequences, which are then associated through one or more disulfide bonds (and with associated light chains). In some embodiments, the signal 1 complex is a non-classical MHC-like molecule such as member of the CD1 family (e.g., CD1a, CD1b, CD1c, CD1d, and CD1e). MHC multimers can be created by direct tethering through peptide or chemical linkers, or can be multimeric via association with streptavidin through biotin moieties. In some embodiments, the antigen presenting complexes are MHC class I or MHC class II molecular complexes involving fusions with immunoglobulin sequences, which are extremely stable and easy to produce, based on the stability and secretion efficiency provided by the immunoglobulin backbone.

MHC class I molecular complexes having immunoglobulin sequences are described in U.S. Patent 6,268,411, which is hereby incorporated by reference in its entirety. These MHC class I molecular complexes may be formed in a conformationally intact fashion at the ends of immunoglobulin heavy chains. MHC class I molecular complexes to which antigenic peptides are bound can stably bind to antigen-specific lymphocyte receptors (e.g., T cell receptors). In various embodiments, the immunoglobulin heavy chain sequence is not full length, but comprises an Ig hinge region, and one or more of CH1, CH2, and/or CH3 domains. The Ig sequence may or may not comprise a variable region, but where variable region sequences are present, the variable region may be full or partial. The complex may further comprise immunoglobulin light chains. MHC class I ligands (e.g., HLA-Ig) lacking variable chain sequences may be employed with site-directed conjugation to particles, as described in WO 2016/105542, which is hereby incorporated by reference in its entirety.

Exemplary MHC class I molecular complexes comprise at least two fusion proteins. A first fusion protein comprises a first MHC class I α chain and a first immunoglobulin heavy chain (or portion thereof comprising the hinge region), and a second fusion protein comprises a second MHC class I α chain and a second immunoglobulin heavy chain (or portion thereof comprising the hinge region). The first and second immunoglobulin heavy chains associate to form the MHC class I molecular complex, which comprises two MHC class I peptide-binding clefts. The immunoglobulin heavy chain can be the heavy chain of an IgM, IgD, IgG1, IgG3, IgG2β, IgG2α, IgG4, IgE, or IgA. In some embodiments, an IgG heavy chain is used to form MHC class I molecular complexes. If multivalent MHC class I molecular complexes are desired, IgM or IgA heavy chains can be used to provide pentavalent or tetravalent molecules, respectively.

Exemplary class I molecules include HLA-A, HLA-B, HLA-C, HLA-E, and these may be employed individually or in any combination. In some embodiments, the antigen presenting complex is an HLA-A2 ligand. The term MHC as used herein, can be replaced by HLA in each instance.

Exemplary MHC class II molecular complexes are described in U.S. Patent 6,458,354, U.S. Patent 6,015,884, U.S. Patent 6,140,113, and U.S. Patent 6,448,071, which are hereby incorporated by reference in their entireties. MHC class II molecular complexes comprise at least four fusion proteins. Two first fusion proteins comprise (i) an immunoglobulin heavy chain (or portion thereof comprising the hinge region) and (ii) an extracellular domain of an MHC class IIβ chain. Two second fusion proteins comprise (i) an immunoglobulin κ or λ light chain (or portion thereof) and (ii) an extracellular domain of an MHC class IIα chain. The two first and the two second fusion proteins associate to form the MHC class II molecular complex. The extracellular domain of the MHC class IIβ chain of each first fusion protein and the extracellular domain of the MHC class IIα chain of each second fusion protein form an MHC class II peptide binding cleft.

The immunoglobulin heavy chain can be the heavy chain of an IgM, IgD, IgG3, IgG1, IgG2β, IgG2α, IgG4, IgE, or IgA. In some embodiments, an IgG1 heavy chain is used to form divalent molecular complexes comprising two antigen binding clefts. Optionally, a variable region of the heavy chain can be included. IgM or IgA heavy chains can be used to provide pentavalent or tetravalent molecular complexes, respectively.

Fusion proteins of an MHC class II molecular complex can comprise a peptide linker inserted between an immunoglobulin chain and an extracellular domain of an MHC class II polypeptide. The length of the linker sequence can vary, depending upon the flexibility required to regulate the degree of antigen binding and receptor cross linking.

Immunoglobulin sequences in some embodiments are humanized monoclonal antibody sequences.

Signal 2 is generally a T cell affecting molecule, that is, a molecule that has a biological effect on a precursor T cell or on an antigen-specific T cell. Such biological effects include, for example, differentiation of a precursor T cell into a CTL, helper T cell (e.g., Th1, Th2), or regulatory T cell; and/or proliferation of T cells. Thus, T cell affecting molecules include T cell costimulatory molecules, adhesion molecules, T cell growth factors, and regulatory T cell inducer molecules. In some embodiments, an aAPC comprises at least one such ligand; optionally, an aAPC comprises at least two, three, or four such ligands.

In certain embodiments, signal 2 is a T cell costimulatory molecule. T cell costimulatory molecules contribute to the activation of antigen-specific T cells. Such molecules include, but are not limited to, molecules that specifically bind to CD28 (including antibodies), CD80 (B7-1), CD86 (B7-2), B7-H3, 4-1BB, 4-1BBL, CD27, CD30, CD134 (OX-40L), B7h (B7RP-1), CD40, LIGHT, antibodies that specifically bind to HVEM, antibodies that specifically bind to CD40L, and antibodies that specifically bind to OX40. In some embodiments, the costimulatory molecule (signal 2) is an antibody (e.g., a monoclonal antibody) or portion thereof, such as F(ab′)2, Fab, scFv, or single chain antibody, or other antigen binding fragment. In some embodiments, the antibody is a humanized monoclonal antibody or portion thereof having antigen-binding activity, or is a fully human antibody or portion thereof having antigen-binding activity.

Combinations of co-stimulatory ligands that may be employed (on the same or separate nanoparticles) include anti-CD28/anti-CD27 and anti-CD28/anti-41BB. The ratios of these co-stimulatory ligands can be varied to effect expansion.

Exemplary signal 1 and signal 2 ligands are described in WO 2014/209868, which describe ligands having a free sulfhydryl (e.g., unpaired cysteine), such that the constant region may be coupled to nanoparticle supports having the appropriate chemical functionality.

Adhesion molecules useful for nano-aAPC can be used to mediate adhesion of the nano-aAPC to a T cell or to a T cell precursor. Useful adhesion molecules include, for example, ICAM-1 and LFA-3.

In some embodiments, signal 1 is provided by peptide-HLA-A2 complexes, and signal 2 is provided by B7.1-Ig or anti-CD28. An exemplary anti-CD28 monoclonal antibody is 9.3 mAb (Tan et al., J. Exp. Med. 1993 177:165), which may be humanized in certain embodiments and/or conjugated to the bead as a fully intact antibody or an antigen-binding fragment thereof.

Some embodiments employ T cell growth factors, which affect proliferation and/or differentiation of T cells. Examples of T cell growth factors include cytokines (e.g., interleukins, interferons) and superantigens. If desired, cytokines can be present in molecular complexes comprising fusion proteins, or can be encapsulated by the aAPC. Particularly useful cytokines include IL-2, IL-4, IL-7, IL-10, IL-12, IL-15, IL-21 gamma interferon, and CXCL10. Optionally, cytokines are provided solely by media components during expansion steps.

The nanoparticles can be made of any material, and materials can be appropriately selected for the desired magnetic property, and may comprise, for example, metals such as iron, nickel, cobalt, or alloy of rare earth metal. Paramagnetic materials also include magnesium, molybdenum, lithium, tantalum, and iron oxide. Paramagnetic beads suitable for enrichment of materials (including cells) are commercially available, and include iron dextran beads, such as dextran-coated iron oxide beads. In aspects of the invention where magnetic properties are not required, nanoparticles can also be made of nonmetal or organic (e.g., polymeric) materials such as cellulose, ceramics, glass, nylon, polystyrene, rubber, plastic, or latex. In exemplary material for preparation of nanoparticles is poly(lactic-co-glycolic acid) (PLGA) or PLA and copolymers thereof, which may be employed in connection with these embodiments. Other materials including polymers and co-polymers that may be employed include those described in PCT/US2014/25889, which is hereby incorporated by reference in its entirety.

In some embodiments, the magnetic particles are biocompatible. This is particularly important in embodiments where the aAPC will be delivered to the patient in association with the enriched and expanded cells. For example, in some embodiments, the magnetic particles are biocompatible iron dextran paramagnetic beads.

In various embodiments, the particle has a size (e.g., average diameter) within about 10 to about 500 nm, or within about 20 to about 200 nm. Especially in embodiments where aAPC will be delivered to patients, microscale aAPC are too large to be carried by lymphatics, and when injected subcutaneously remain at the injection site. When injected intravenously, they lodge in capillary beds. In fact, the poor trafficking of microscale beads has precluded the study of where aAPC should traffic for optimal immunotherapy. Trafficking and biodistribution of nano-aAPC is likely to be more efficient than microscale aAPC. For example, two potential sites where an aAPC might be most effective are the lymph node, where naive and memory T cells reside, and the tumor itself. Nanoparticles of about 50 to about 200 nm diameter can be taken up by lymphatics and transported to the lymph nodes, thus gaining access to a larger pool of T cells. As described in PCT/US2014/25889, which is hereby incorporated by reference, subcutaneous injection of nano-aAPCs resulted in less tumor growth than controls or intravenously injected beads.

For magnetic clustering, it is preferred that the nanoparticles have a size in the range of 10 to 250 nm, or 20 to 100 nm in some embodiments. Receptor-ligand interactions at the cell-nanoparticle interface are not well understood. However, nanoparticle binding and cellular activation are sensitive to membrane spatial organization, which is particularly important during T cell activation, and magnetic fields can be used to manipulate cluster-bound nanoparticles to enhance activation. For example, T cell activation induces a state of persistently enhanced nanoscale TCR clustering and nanoparticles are sensitive to this clustering in a way that larger particles are not.

Furthermore, nanoparticle interactions with TCR clusters can be exploited to enhance receptor triggering. T cell activation is mediated by aggregation of signaling proteins, with “signaling clusters” hundreds of nanometers across, initially forming at the periphery of the T cell-APC contact site and migrating inward. As described herein, an external magnetic field can be used to enrich antigen-specific T cells (including rare naive cells) and to drive aggregation of magnetic nano-aAPC bound to TCR, resulting in aggregation of TCR clusters and enhanced activation of naïve T cells. Magnetic fields can exert appropriately strong forces on paramagnetic particles, but are otherwise biologically inert, making them a powerful tool to control particle behavior. T cells bound to paramagnetic nano-aAPC are activated in the presence of an externally applied magnetic field. Nano-aAPC are themselves magnetized, and attracted to both the field source and to nearby nanoparticles in the field, inducing bead and thus TCR aggregation to boost aAPC-mediated activation.

Nano-aAPCs bind more TCR on and induce greater activation of previously activated compared to naive T cells. In addition, application of an external magnetic field induces nano-aAPC aggregation on naive cells, enhancing T cells proliferation both in vitro and following adoptive transfer in vivo. Importantly, in a melanoma adoptive immunotherapy model, T cells activated by nano-aAPC in a magnetic field mediate tumor rejection. Thus, the use of applied magnetic fields permits activation of naive T cell populations, which otherwise are poorly responsive to stimulation. This is an important feature of immunotherapy as naive T cells have been shown to be more effective than more differentiated subtypes for cancer immunotherapy, with higher proliferative capacity and greater ability to generate strong, long-term T cell responses. Thus, nano-aAPC can used for magnetic field enhanced activation of T cells to increase the yield and activity of antigen-specific T cells expanded from naive precursors, improving cellular therapy for, e.g., patients with infectious diseases, cancer, or autoimmune diseases, or to provide prophylactic protection to immunosuppressed patients.

Molecules can be directly attached to nanoparticles by adsorption or by direct chemical bonding, including covalent bonding. See, Hermanson, BIOCONJUGATE TECHNIQUES, Academic Press, New York, 1996. A molecule itself can be directly activated with a variety of chemical functionalities, including nucleophilic groups, leaving groups, or electrophilic groups. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, a molecule can be bound to a nanoparticle through the use of a small molecule-coupling reagent. Non-limiting examples of coupling reagents include carbodiimides, maleimides, n-hydroxysuccinimide esters, bischloroethylamines, bifunctional aldehydes such as glutaraldehyde, anyhydrides and the like. In other embodiments, a molecule can be coupled to a nanoparticle through affinity binding such as a biotin-streptavidin linkage or coupling, as is well known in the art. For example, streptavidin can be bound to a nanoparticle by covalent or non-covalent attachment, and a biotinylated molecule can be synthesized using methods that are well known in the art.

If covalent binding to a nanoparticle is contemplated, the support can be coated with a polymer that contains one or more chemical moieties or functional groups that are available for covalent attachment to a suitable reactant, typically through a linker. For example, amino acid polymers can have groups, such as the ε-amino group of lysine, available to couple a molecule covalently via appropriate linkers. This disclosure also contemplates placing a second coating on a nanoparticle to provide for these functional groups.

Activation chemistries can be used to allow the specific, stable attachment of molecules to the surface of nanoparticles. There are numerous methods that can be used to attach proteins to functional groups. For example, the common cross-linker glutaraldehyde can be used to attach protein amine groups to an aminated nanoparticle surface in a two-step process. The resultant linkage is hydrolytically stable. Other methods include use of cross-linkers containing n-hydrosuccinimido (NHS) esters which react with amines on proteins, cross-linkers containing active halogens that react with amine-, sulfhydryl-, or histidine-containing proteins, cross-linkers containing epoxides that react with amines or sulfhydryl groups, conjugation between maleimide groups and sulfhydryl groups, and the formation of protein aldehyde groups by periodate oxidation of pendant sugar moieties followed by reductive amination.

In some embodiments, signal 1 and/or signal 2 ligands are chemically conjugated to particles through a free cysteine engineered in the Fc region of immunoglobulin sequences.

The ratio of particular ligands when used simultaneously on the same or different particles can be varied to increase the effectiveness of the nanoparticle in antigen or costimulatory ligand presentation. For example, nanoparticles can be coupled with HLA-A2-Ig and anti-CD28 (or other signal 2 ligands) at a variety of ratios, such as about 30:1, about 25:1, about 20:1, about 15:1, about 10:1, about 5:1, about 3:1, about 2:1, about 1:1, about 0.5:1, about 0.3:1; about 0.2:1, about 0.1:1, or about 0.03:1. In some embodiments, the ratio is from 2:1 to 1:2. The total amount of protein coupled to the supports may be, for example, about 250 mg/ml, about 200 mg/ml, about 150 mg/ml, about 100 mg/ml, or about 50 mg/ml of particles. Because effector functions such as cytokine release and growth may have differing requirements for Signal 1 versus Signal 2 than T cell activation and differentiation, these functions can be determined separately.

The configuration of nanoparticles can vary from being irregular in shape to being spherical and/or from having an uneven or irregular surface to having a smooth surface. Non-spherical aAPCs are described in WO 2013/086500, which is hereby incorporated by reference in its entirety.

In certain embodiments, the aAPCs are paramagnetic particles in the range of 50 to 100 nm (e.g., approximately 85 nm), with a PDI (size distribution) of less than 0.2, or in some embodiments less than 0.1. The aAPCs may have a surface charge of from 0 to -10 mV, such as from about -2 to -6 mV. aAPCs may have from 10 to 120 ligands per particle, such as from about 25 to about 100 ligands per particle, with ligands conjugated to the particle through a free cysteine introduced in the Fc region of the immunoglobulin sequences. The particles may contain about 1:1 ratio of HLA dimer:anti-CD28, which may be present on the same or different populations of particles. The nanoparticles provide potent expansion of cognate T cells, while exhibiting no stimulation of non-cognate TCRs, even with passive loading of peptide antigen. Particles are stable in lyophilized form for at least two or three years.

The aAPCs present antigen to T cells and thus can be used to both enrich for and expand antigen-specific T cells, including from naive T cells. The peptide antigens will be selected based on the desired therapy, for example, cancer, type of cancer, infectious disease, etc. In some embodiments, the method is conducted to treat a cancer patient, and neoantigens specific to the patient are identified, and synthesized for loading aAPCs. In some embodiments, between three and ten neoantigens are identified through genetic analysis of the tumor (e.g., nucleic acid sequencing), followed by predictive bioinformatics. As shown herein, several antigens can be employed together (on separate aAPCs), with no loss of functionality in the method. In some embodiments, the antigens are natural, non-mutated, cancer antigens, of which many are known. This process for identifying antigens on a personalized basis is described in greater detail below.

A variety of antigens can be bound to antigen presenting complexes. The nature of the antigens depends on the type of antigen presenting complex that is used. For example, peptide antigens can be bound to MHC class I and class II peptide binding clefts. Non-classical MHC-like molecules can be used to present non-peptide antigens such as phospholipids, complex carbohydrates, and the like (e.g., bacterial membrane components such as mycolic acid and lipoarabinomannan). Any peptide capable of inducing an immune response can be bound to an antigen presenting complex. Antigenic peptides include tumor-associated antigens, autoantigens, alloantigens, and antigens of infectious agents.

“Tumor-associated antigens” include unique tumor antigens expressed exclusively by the tumor from which they are derived, shared tumor antigens expressed in many tumors but not in normal adult tissues (oncofetal antigens), and tissue-specific antigens expressed also by the normal tissue from which the tumor arose. Tumor associated antigens can be, for example, embryonic antigens, antigens with abnormal post-translational modifications, differentiation antigens, products of mutated oncogenes or tumor suppressors, fusion proteins, or oncoviral proteins.

A variety of tumor-associated antigens are known in the art, and many of these are commercially available. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastriccarcinoma), alphafetoprotein receptor (expressed in multiple tumor types, particularly mammary tumors), and M2A (expressed in germ cell neoplasia).

Tumor-associated differentiation antigens include tyrosinase (expressed in melanoma) and particular surface immunoglobulins (expressed in lymphomas).

Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer), ras, p53, nonmutant associated with breast tumors, MAGE-1, and MAGE-3 (associated with melanoma, lung, and other cancers). Fusion proteins include BCR-ABL, which is expressed in chromic myeloid leukemia. Oncoviral proteins include HPV type 16, E6, and E7, which are found in cervical carcinoma.

Tissue-specific antigens include melanotransferrin and MUC1 (expressed in pancreatic and breast cancers); CD10 (previously known as common acute lymphoblastic leukemia antigen, or CALLA) or surface immunoglobulin (expressed in B cell leukemias and lymphomas); the α chain of the IL-2 receptor, T cell receptor, CD45R, CD4+/CD8+ (expressed in T cell leukemias and lymphomas); prostatespecific antigen and prostatic acid-phosphatase (expressed in prostate carcinoma); GP 100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

For example, in some embodiments, the patient to be treated has bladder cancer, and T cells are enriched and expanded with one or more of NY-ESO-1, MAGE-A10, and MUC-1 antigens. In some embodiments, the patient to be treated has brain cancer, and T cells are enriched and expanded with one or more of NY-ESO-1, Survivin, and CMV antigens. In some embodiments, the patient to be treated has breast cancer, and T cells are enriched and expanded with one or more of MUC-1, Surivin, WT-1, HER-2, and CEA antigens. In some embodiments, the patient to be treated has cervical cancer, and T cells are enriched and expanded with HPV antigen. In some embodiments, the patient to be treated has colorectal cancer, and T cells are enriched and expanded with one or more of NY-ESO-1, Survivin, WT-1, MUC-1, and CEA antigens. In some embodiments, the patient to be treated has esophageal cancer, and T cells are enriched and expanded with NY-ESO-1 antigen. In some embodiments, the patient to be treated has head and neck cancer, and T cells are enriched and expanded with HPV antigen. In some embodiments, the patient to be treated has kidney or liver cancer, and T cells are enriched and expanded with NY-ESO-1 antigen. In some embodiments, the patient to be treated has lung cancer, and T cells are enriched and expanded with one or more of NY-ESO-1, Survivin, WT-1, MAGE-A10, and MUC-1 antigens. In some embodiments, the patient to be treated has melanoma, and T cells are enriched and expanded with one or more of NY-ESO-1, Survivin, MAGE-A10, MART-1, and GP-100. In some embodiments, the patient to be treated has ovarian cancer, and T cells are enriched and expanded with one or more of NY-ESO-1, WT-1, and Mesothelin antigen. In some embodiments, the patient to be treated has prostate cancer, and T cells are enriched and expanded with one or more of Survivin, hTERT, PSA, PAP, and PSMA antigens. In some embodiments, the patient to be treated has a sarcoma, and T cells are enriched and expanded with NY-ESO-1 antigen. In some embodiments, the patient to be treated has lymphoma, and T cells are enriched and expanded with EBV antigen. In some embodiments, the patient to be treated has multiple myeloma, and T cells are enriched and expanded with one or more of NY-ESO-1, WT-1, and SOX2 antigens. In some embodiments, the patient to be treated has lymphoma, and T cells are enriched and expanded with EBV antigen.

In some embodiments, the patient to be treated has acute myelogenous leukemia or myelodysplastic syndrome, and T cells are enriched and expanded with one or more of (including 1, 2, 3, 4, or 5 of) Survivin, WT-1, PRAME, RHAMM and PR3 antigens.

“Antigens of infectious agents” include components of protozoa, bacteria, fungi (both unicellular and multicellular), viruses, prions, intracellular parasites, helminths, and other infectious agents that can induce an immune response.

Bacterial antigens include antigens of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

Antigens of protozoan infectious agents include antigens of malarial plasmodia, Leishmania species, Trypanosoma species and Schistosoma species.

Fungal antigens include antigens of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

Viral peptide antigens include, but are not limited to, those of adenovirus, herpes simplex virus, papilloma virus, respiratory syncytial virus, poxviruses, HIV, influenza viruses, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

Antigens, including antigenic peptides, can be bound to an antigen binding cleft of an antigen presenting complex either actively or passively, as described in U.S. Patent 6,268,411, which is hereby incorporated by reference in its entirety. Optionally, an antigenic peptide can be covalently bound to a peptide binding cleft.

If desired, a peptide tether can be used to link an antigenic peptide to a peptide binding cleft. For example, crystallographic analyses of multiple class I MHC molecules indicate that the amino terminus of β2M is very close, approximately 20.5 Angstroms away, from the carboxyl terminus of an antigenic peptide resident in the MHC peptide binding cleft. Thus, using a relatively short linker sequence, approximately 13 amino acids in length, one can tether a peptide to the amino terminus of β2M. If the sequence is appropriate, that peptide will bind to the MHC binding groove (see U.S. Patent 6,268,411).

Antigen-specific T cells which are bound to the aAPCs can be separated from cells which are not bound using magnetic enrichment, or other cell sorting or capture technique. Other processes that can be used for this purpose include flow cytometry and other chromatographic means (e.g., involving immobilization of the antigen-presenting complex or other ligand described herein). In one embodiment antigen-specific T cells are isolated (or enriched) by incubation with beads, for example, antigen-presenting complex/anti-CD28-conjugated paramagnetic beads (such as DYNABEADS®), for a time period sufficient for positive selection of the desired antigen-specific T cells.

In some embodiments, a population of T cells can be substantially depleted of previously active T cells using, e.g., an antibody to CD44, leaving a population enriched for naïve T cells. Binding nano-aAPCs to this population would not substantially activate the naïve T cells, but would permit their purification.

In still other embodiments, ligands that target NK cells, NKT cells, or B cells (or other immune effector cells), can be incorporated into a paramagnetic nanoparticle, and used to magnetically enrich for these cell populations, optionally with expansion in culture as described below. Additional immune effector cell ligands are described in PCT/US2014/25889, which is hereby incorporated by reference in its entirety.

Without wishing to be bound by theory, removal of unwanted cells may reduce competition for cytokines and growth signals, remove suppressive cells, or may simply provide more physical space for expansion of the cells of interest.

Enriched T cells are then expanded in culture optionally within the proximity of a magnet for a period of time to produce a magnetic field, which enhances T cell receptor clustering of aAPC bound cells. Cultures can be stimulated for variable amounts of time, such as from about 5 minutes to about 72 hours (e.g., about 0.5, 2, 6, 12, 36, 48, or 72 hours as well as continuous stimulation) with nano-aAPC. The effect of stimulation time in highly enriched antigen-specific T cell cultures can be assessed. Antigen-specific T cell can be placed back in culture and analyzed for cell growth, proliferation rates, various effector functions, and the like, as is known in the art. Such conditions may vary depending on the antigen-specific T cell response desired. In some embodiments, T cells are expanded in culture from about 2 days to about 3 weeks, or in some embodiments, about 5 days to about 2 weeks, or about 5 days to about 10 days. In some embodiments, the T cells are expanded in culture for about 1 week, after which time a second enrichment and expansion step is optionally performed. In some embodiments, 2, 3, 4, or 5 enrichment and expansion rounds are performed.

After the one or more rounds of enrichment and expansion (e.g. about 7 days), the antigen-specific T cell component of the sample will be at least about 1% of the T cells, or in some embodiments, at least about 5%, at least about 10%, at least about 15%, or at least about 20%, or at least about 25% of the T cells in the sample. Further, these T cells generally display an activated state. From the original sample isolated from the patient, the antigen-specific T cells in various embodiments are expanded (in about 7 days) from about 100-fold to about 10,000 fold, such as at least about 100-fold, or at least about 200-fold. After 2 weeks, antigen-specific T cells are expanded at least 1000-fold, or at least about 2000-fold, at least about 3,000 fold, at least about 4,000-fold, or at least about 5,000-fold in various embodiments. In some embodiments, antigen-specific T cells are expanded by greater than 5000-fold or greater than 10,000 fold after two weeks. After the one or more rounds of enrichment and expansion (one or two weeks), at least about 10⁶, or at least about 10⁷, or at least about 10⁸, or at least about 10⁹ antigen-specific T cells are obtained.

The effect of nano-aAPC on expansion, activation and differentiation of T cell precursors can be assayed in any number of ways known to those of skill in the art. A rapid determination of function can be achieved using a proliferation assay, by determining the increase of CTL, helper T cells, or regulatory T cells in a culture by detecting markers specific to each type of T cell. Such markers are known in the art. CTL can be detected by assaying for cytokine production or for cytolytic activity using chromium release assays.

In addition to generating antigen-specific T cells with appropriate effector functions, another parameter for antigen-specific T cell efficacy is expression of homing receptors that allow the T cells to traffic to sites of pathology (Sallusto et al., Nature 401, 708-12, 1999; Lanzavecchia & Sallusto, Science 290, 92-97, 2000).

For example, effector CTL efficacy has been linked to the following phenotype of homing receptors, CD62L+, CD45RO+, and CCR7-. Thus, a nano-aAPC-induced and/or expanded CTL population can be characterized for expression of these homing receptors. Homing receptor expression is a complex trait linked to initial stimulation conditions. Presumably, this is controlled both by the co-stimulatory complexes as well as cytokine milieu. One important cytokine that has been implicated is IL-12 (Salio et al., 2001). As discussed below, nano-aAPC offer the potential to vary individually separate components (e.g., T cell effector molecules and antigen presenting complexes) to optimize biological outcome parameters. Optionally, cytokines such as IL-12 can be included in the initial induction cultures to affect homing receptor profiles in an antigen- specific T cell population.

Optionally, a cell population comprising antigen-specific T cells can continue to be incubated with either the same nano-aAPC or a second nano-aAPC for a period of time sufficient to form a second cell population comprising an increased number of antigen-specific T cells relative to the number of antigen-specific T cells in the first cell population. Typically, such incubations are carried out for 3-21 days, preferably 7-10 days.

Suitable incubation conditions (culture medium, temperature, etc.) include those used to culture T cells or T cell precursors, as well as those known in the art for inducing formation of antigen-specific T cells using DC or artificial antigen presenting cells. See, e.g., Latouche & Sadelain, Nature Biotechnol.18, 405-09, April 2000; Levine et al., J. Immunol. 159, 5921-30, 1997; Maus et al., Nature Biotechnol. 20, 143-48, February 2002. See also the specific examples, below.

To assess the magnitude of a proliferative signal, antigen-specific T cell populations can be labeled with CFSE and analyzed for the rate and number of cell divisions. T cells can be labeled with CFSE after one-two rounds of stimulation with nano-aAPC to which an antigen is bound. At that point, antigen-specific T cells should represent 2-10% of the total cell population. The antigen-specific T cells can be detected using antigen-specific staining so that the rate and number of divisions of antigen-specific T cells can be followed by CFSE loss. At varying times (for example, 12, 24, 36, 48, and 72 hours) after stimulation, the cells can be analyzed for both antigen presenting complex staining and CFSE. Stimulation with nano-aAPC to which an antigen has not been bound can be used to determine baseline levels of proliferation. Optionally, proliferation can be detected by monitoring incorporation of 3H-thymidine, as is known in the art.

Antigen-specific T cells obtained using nano-aAPC, can be administered to patients by any appropriate routes, including intravenous administration, intra-arterial administration, subcutaneous administration, intradermal administration, intralymphatic administration, and intratumoral administration. Patients include both human and veterinary patients.

Antigen-specific regulatory T cells can be used to achieve an immunosuppressive effect, for example, to treat or prevent graft versus host disease in transplant patients, or to treat or prevent autoimmune diseases, such as those listed above, or allergies. Uses of regulatory T cells are disclosed, for example, in US 2003/0049696, US 2002/0090724, US 2002/0090357, US 2002/0034500, and US 2003/0064067, which are hereby incorporated by reference in their entireties.

Antigen-specific T cells prepared according to these methods can be administered to patients in doses ranging from about 5-10 × 10⁶ CTL/kg of body weight (~7 ×10⁸ CTL/treatment) up to about 3.3 × 10⁹CTL/kg of body weight (~6 × 10⁹ CTL/treatment) (Walter et al., New England Journal of Medicine 333, 1038-44, 1995; Yee et al., J Exp Med 192, 1637-44, 2000). In other embodiments, patients can receive about 10³, about 5 × 10³, about 10⁴, about 5 × 10⁴, about 10⁵, about 5 × 10⁵, about 10⁶, about 5 × 10⁶, about 10⁷, about 5 × 10⁷, about 10⁸, about 5 × 10⁸, about 10⁹, about 5 × 10⁹, or about 10¹⁰ cells per dose administered intravenously. In still other embodiments, patients can receive intranodal injections of, e.g., about 8 × 10⁶ or about 12 × 10⁶ cells in a 200 µl bolus. Doses of nano-APC that are optionally administered with cells include at least about 10³, about 5 × 10³, about 10⁴, about 5 × 10⁴, about 10⁵, about 5 × 10⁵, about 10⁶, about 5 × 10⁶, about 10⁷, about 5 × 10⁷, about 10⁸, about 5 × 10⁸, about 10⁹, about 5 × 10⁹, about 10¹⁰, about 5 × 10¹⁰, about 10¹¹, about 5 × 10¹¹, or about 10¹² nano-aAPC per dose.

In an exemplary embodiment, the enrichment and expansion process is performed repeatedly on the same sample derived from a patient. A population of T cells is enriched and activated on Day 0, followed by a suitable period of time (e.g., about 3-20 days) in culture. Subsequently, nano-aAPC can be used to again enrich and expand against the antigen of interest, further increasing population purity and providing additional stimulus for further T cell expansion. The mixture of nano-aAPC and enriched T cells may subsequently again be cultured in vitro for an appropriate period of time, or immediately re-infused into a patient for further expansion and therapeutic effect in vivo. Enrichment and expansion can be repeated any number of times until the desired expansion is achieved.

In some embodiments, a cocktail of nano-aAPC, each against a different antigen, can be used at once to enrich and expand antigen T cells against multiple antigens simultaneously. In this embodiment, a number of different nano-aAPC batches, each bearing a different MHC-peptide, would be combined and used to simultaneously enrich T cells against each of the antigens of interest. The resulting T cell pool would be enriched and activated against each of these antigens, and responses against multiple antigens could thus be cultured simultaneously. These antigens could be related to a single therapeutic intervention; for example, multiple antigens present on a single tumor.

In some embodiments, the patient receives immunotherapy with one or more checkpoint inhibitors, prior to receiving the antigen-specific T cells by adoptive transfer, or prior to direct administration of aAPCs bearing neoantigens identified in vitro through genetic analysis of the patient’s tumor. In various embodiments, the checkpoint inhibitor(s) target one or more of CTLA-4 or PD-1/PD-L1, which may include antibodies against such targets, such as monoclonal antibodies, or portions thereof, or humanized or fully human versions thereof. In some embodiments, the checkpoint inhibitor therapy comprises ipilimumab or Keytruda (pembrolizumab).

In some embodiments, the patient receives about 1 to 5 rounds of adoptive immunotherapy (e.g., one, two, three, four or five rounds). In some embodiments, each administration of adoptive immunotherapy is conducted simultaneously with, or after (e.g., from about 1 day to about 1 week after), a round of checkpoint inhibitor therapy. In some embodiments, adoptive immunotherapy is provided about 1 day, about 2 days, about 3 days, about 4 days, about 5 days, about 6 days, or about 1 week after a checkpoint inhibitor dose.

In still other embodiments, adoptive transfer or direct infusion of nano-aAPCs to the patient comprises, as a ligand on the bead, a ligand that targets one or more of CTLA-4 or PD-1/PD-L1. In these embodiments, the method can avoid certain side effects of administering soluble checkpoint inhibitor therapy.

In some aspects, the invention provides methods for personalized cancer immunotherapy. The methods are accomplished using the aAPCs to identify antigens to which the patient will respond, followed by administration of the appropriate peptide-loaded aAPC to the patient, or followed by enrichment and expansion of the antigen specific T cells ex vivo.

Genome-wide sequencing has dramatically altered our understanding of cancer biology. Sequencing of cancers has yielded important data regarding the molecular processes involved in the development of many human cancers. Driving mutations have been identified in key genes involved in pathways regulating three main cellular processes (1) cell fate, (2) cell survival and (3) genome maintenance. Vogelstein et al., Science 339, 1546-58 (2013).

Genome-wide sequencing also has the potential to revolutionize our approach to cancer immunotherapy. Sequencing data can provide information about both shared as well as personalized targets for cancer immunotherapy. In principle, mutant proteins are foreign to the immune system and are putative tumor-specific antigens. Indeed, sequencing efforts have defined hundred if not thousands of potentially relevant immune targets. Limited studies have shown that T cell responses against these neo-epitopes can be found in cancer patients or induced by cancer vaccines. However, the frequency of such responses against a particular cancer and the extent to which such responses are shared between patients are not well known. One of the main reasons for our limited understanding of tumor-specific immune responses is that current approaches for validating potential immunologically relevant targets are cumbersome and time consuming.

Thus, in some aspects, the invention provides a high-throughput platform-based approach for detection of T cell responses against neo-antigens in cancer. This approach uses the aAPC platform described herein for the detection of even low-frequency T cell responses against cancer antigens. Understanding the frequency and between-person variability of such responses would have important implications for the design of cancer vaccines and personalized cancer immunotherapy.

Although central tolerance abrogates T cell responses against self-proteins, oncogenic mutations induce neo-epitopes against which T cell responses can form. Mutation catalogues derived from whole exome sequencing provide a starting point for identifying such neo-epitopes. Using HLA binding prediction algorithms (Srivastava, PLoS One 4, e6094 (2009), it has been predicted that each cancer can have up 7-10 neo-epitopes. A similar approach estimated hundreds of tumor neo-epitopes. Such algorithms, however, may have low accuracy in predicting T cell responses, and only 10% of predicted HLA-binding epitopes are expected to bind in the context of HLA (Lundegaard C, Immunology 130, 309-18 (2010)). Thus, predicted epitopes must be validated for the existence of T cell responses against those potential neo-epitopes.

In certain embodiments, the nano-aAPC system is used to screen for neo-epitopes that induce a T cell response in a variety of cancers, or in a particular patient’s cancer. Cancers may be genetically analyzed, for example, by whole exome-sequencing. For example, of a panel of 24 advanced adenocarcinomas, an average of about 50 mutations per tumor were identified. Of approximately 20,000 genes analyzed, 1327 had at least one mutation, and 148 had two or more mutations. 974 missense mutations were identified, with a small additional number of deletions and insertions.

A list of candidate peptides can be generated from overlapping nine amino acid windows in mutated proteins. All nine-AA windows that contain a mutated amino acid, and 2 non-mutated “controls” from each protein will be selected. These candidate peptides will be assessed computationally for MHC binding using a consensus of MHC binding prediction algorithms, including Net MHC and stabilized matrix method (SMM). Nano-aAPC and MHC binding algorithms have been developed primarily for HLA-A2 allele. The sensitivity cut-off of the consensus prediction can be adjusted until a tractable number of mutation containing peptides (~500) and non-mutated control peptides (~50) are identified.

A peptide library is then synthesized. MHC (e.g., A2) bearing aAPC are deposited in multi well plates and passively loaded with peptide. CD8 T cells may be isolated from PBMC of both A2 positive healthy donors and A2 positive cancers patients. Subsequently, the isolated T cells are incubated with the loaded aAPCs for the enrichment step. Following the incubation, the plates or culture flasks are placed on a magnetic field and the supernatant containing irrelevant T cells not bound to the aAPCs is removed. The remaining T cells that are bound to the aAPCs will be cultured and allowed to expand for 7 to 21 days. Antigen specific expansion is assessed by re-stimulation with aAPC and intracellular IFNγ fluorescent staining.

In some embodiments, a patient’s T cells are screened against an array or library of nanoAPCs, and the results are used for diagnostic or prognostic purposes. For example, the number and identity of T cell anti-tumor responses against mutated proteins, overexpressed proteins, and/or other tumor-associated antigens can be used as a biomarker to stratify risk. For example, the number of such T cell responses may be inversely proportionate to the risk of disease progression or risk of resistance or non-responsiveness to chemotherapy. In other embodiments, the patient’s T cells are screened against an array or library of nano-APCs, and the presence of T cells responses, or the number or intensity of these T cells responses identifies that the patient has a sub-clinical tumor, and/or provides an initial understanding of the tumor biology.

In some embodiments, a patient or subject’s T cells are screened against an array or library of paramagnetic aAPCs, each presenting a different candidate peptide antigen. This screen can provide a wealth of information concerning the subject or patient’s T cell repertoire, and the results are useful for diagnostic or prognostic purposes. For example, the number and identity of T cell anti-tumor responses against mutated proteins, overexpressed proteins, and/or other tumor-associated antigens can be used as a biomarker to stratify risk, to monitor efficacy of immunotherapy, or predict outcome of immunotherapy treatment. Further, the number or intensity of such T cell responses may be inversely proportionate to the risk of disease progression or may be predictive of resistance or non-responsiveness to chemotherapy. In other embodiments, a subject’s or patient’s T cells are screened against an array or library of nano-APCs each presenting a candidate peptide antigen, and the presence of T cells responses, or the number or intensity of these T cells responses, provides information concerning the health of the patient, for example, by identifying autoimmune disease, or identifying that the patient has a sub-clinical tumor. In these embodiments, the process not only identifies a potential disease state, but provides an initial understanding of the disease biology.

In an exemplary embodiment, the patient has a hematological cancer such as acute myelogenous leukemia (AML) or myelodysplastic syndrome, and in some embodiments the patient has relapsed after allogeneic stem cell transplantation. Using a source of T cells from an HLA matched donor, antigen-specific T cells are magnetically enriched and activated using a magnetic column and paramagnetic nano-aAPC presenting from 2 to 5 tumor associated peptide antigens, which are optionally selected from Survivin, WT-1, PRAME, RHAMM, and PR3. The antigens are passively loaded onto prepared nano-aAPCs, which present signal 1 and signal 2 on the same or different populations of particles through site-directed conjugation.

Magnetic activation may take place for from 5 minutes to 5 hours, or from 5 minutes to 2 hours, followed by expansion in culture for at least 5 days, and up to 2 weeks or up to 3 weeks in some embodiments. Resulting CD8+ T cells may be phenotypically characterized to confirm: low PD-1 expression; central memory phenotype (CD3+, CD45RA-, CD62L+); and effector memory phenotype (CD3+, CD45RA-, CD62L-). Expanded T cells can be administered to the patient at from 1 to about 4 administrations, to establish an anti-tumor response.

Other aspects and embodiments of the present invention will be apparent to the skilled artisan based on the following illustrative examples.

### EXAMPLES

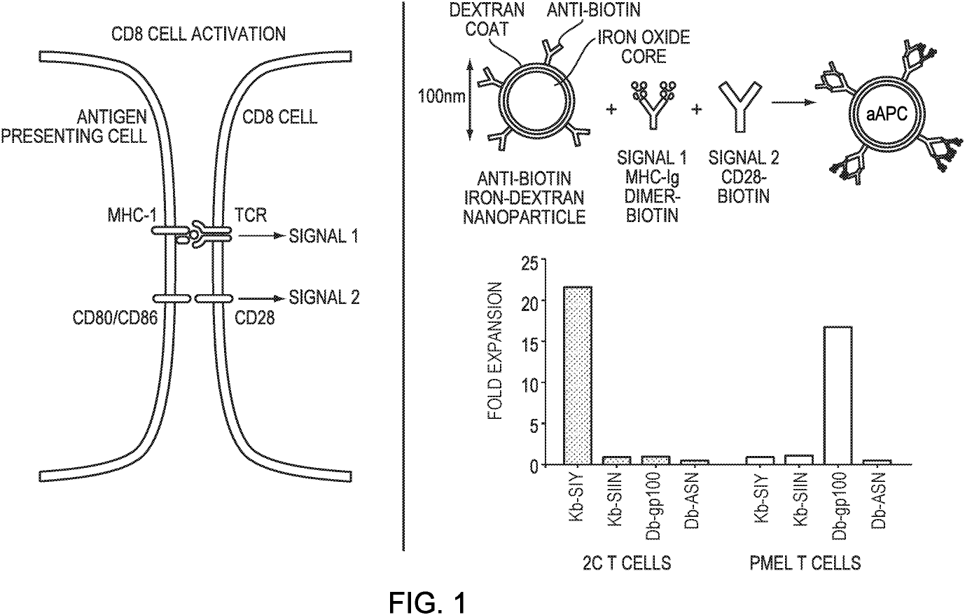

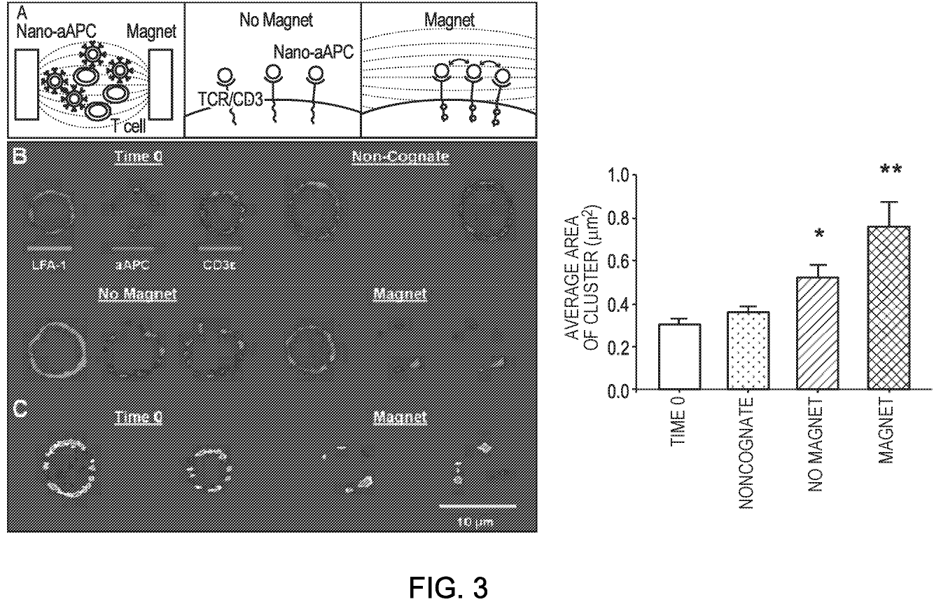

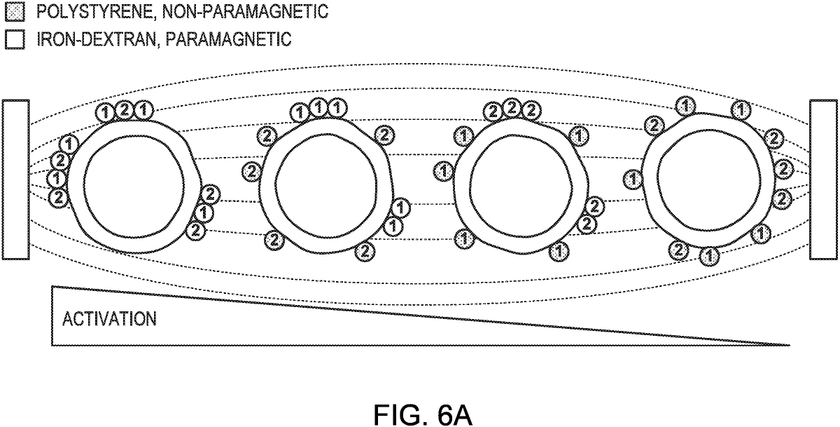

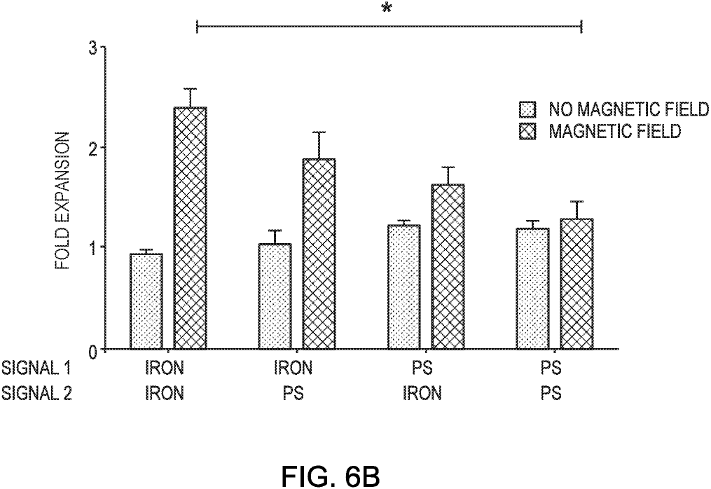

Artificial Antigen Presenting Cells (aAPCs) can be constructed on paramagnetic particles, such as dextran-coated iron oxide nanoparticles, for activation of antigen-specific T cells. FIG. 1. The presence of a signal 1, with a signal 2, results in T cell activation and expansion. By using paramagnetic particles, clustering of signal 1 and/or signal 2 can be induced by a magnetic field. FIG. 3 and FIG. 6A.

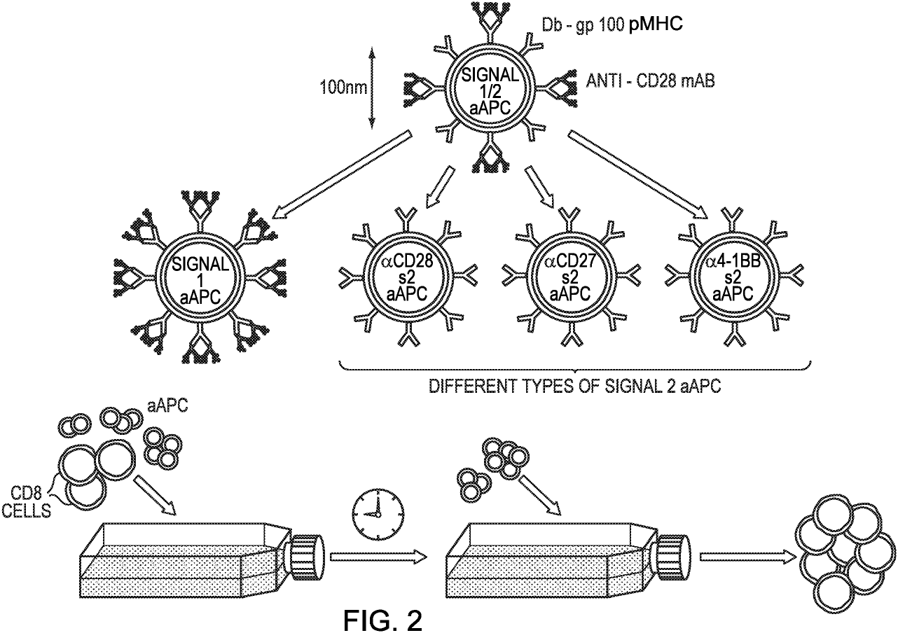

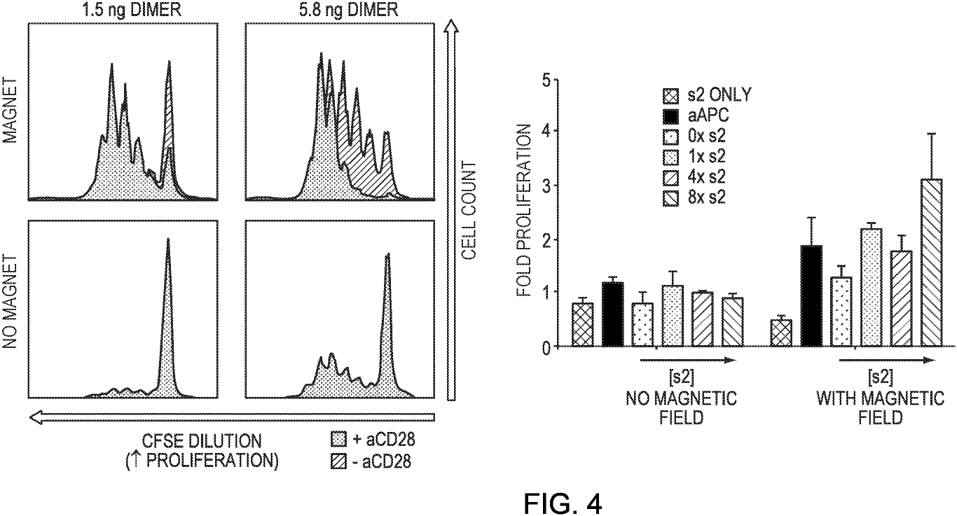

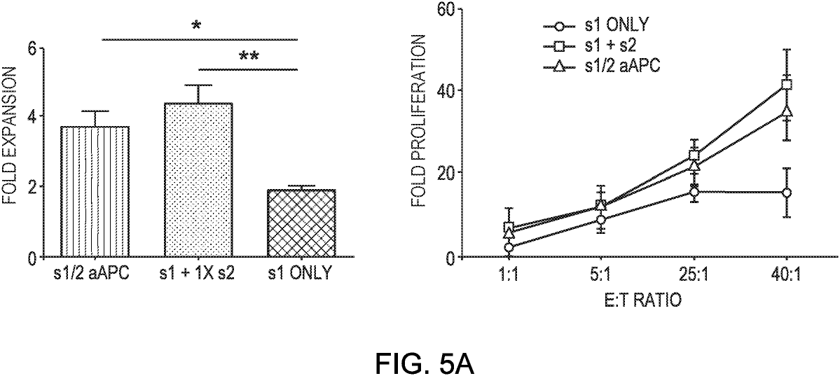

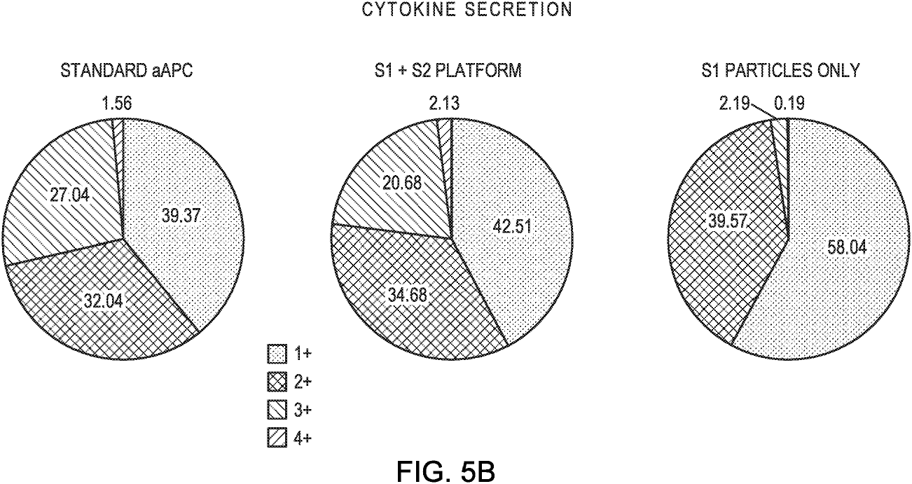

By controlling the presentation of the co-stimulatory signal (signal 2) on separate nanoparticles, the type of co-stimulatory signal can be controlled and varied. FIG. 2. The presence of a magnetic field when using paramagnetic particles enhances proliferation of T cells, and this effect is dependent on the amount of signal 2 present on separate nanoparticles from signal 1. FIG. 4. The resultant T cells, whether signal 1 and 2 are present on the same or different particles, are qualitatively the same. FIG. 5.

The highest expansion of antigen-specific T cells was observed when both signal 1 and signal 2 were present on separate (paramagnetic or non-paramagnetic) beads, with the highest expansion observed when both particles are paramagnetic. FIG. 6B.

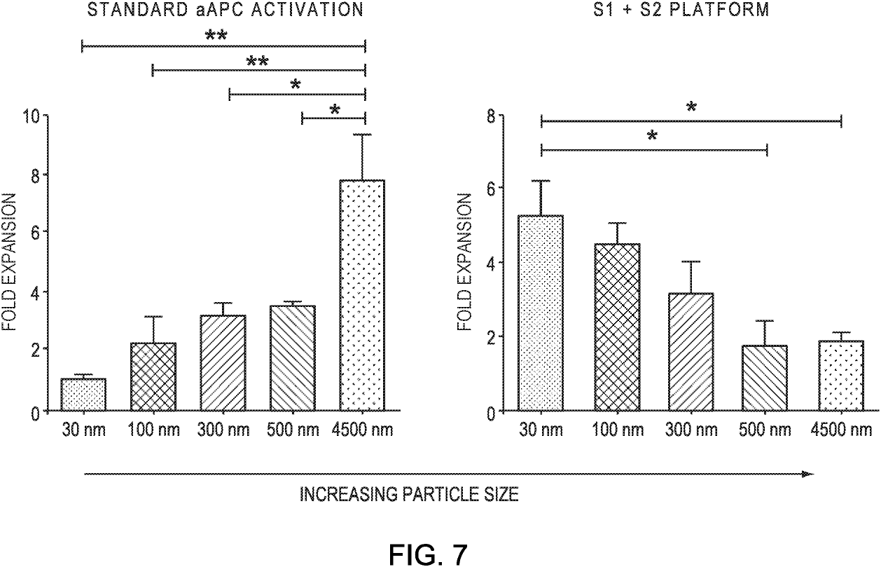

As particle size increases, the efficacy of the S1+S2 approach decreases. In contrast, nanoparticles containing both signals show the opposite effect. Thus, when using separate nanoparticles for signal 1 and signal 2, particles are preferably kept at 200 nm or less, such as 100 nm or 30 nm, which supported high levels of expansion. FIG. 7.

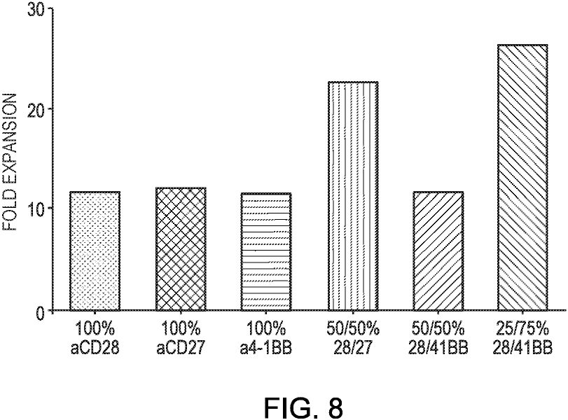

The types of co-stimulation can be varied to customize the activation profile, by placing each signal on a separate bead. For example, signal 2 beads containing 50/50 anti-CD28 and anti-CD27, as well as signal 2 beads containing 25/75 anti-CD28 and anti-41BB, supported high levels of expansion. FIG. 8.

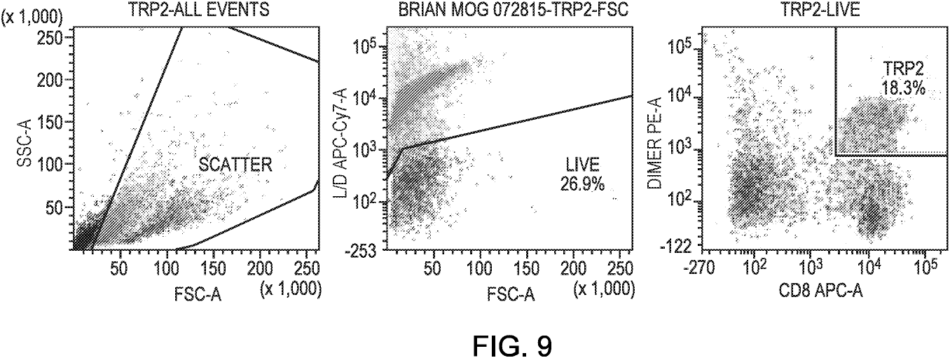

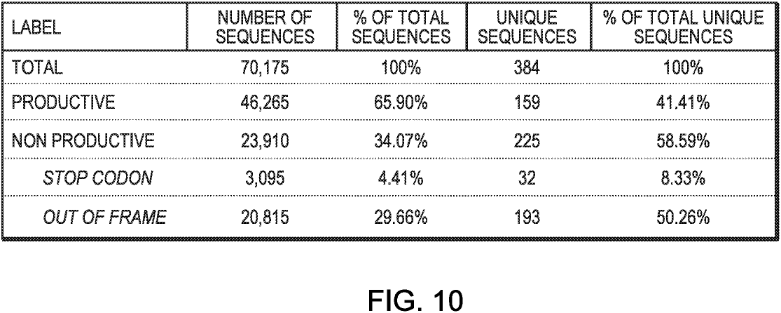

Magnetic enrichment and expansion lead to rapid identification of novel TCRs with desired antigen specificity. FIG. 9, FIG. 10. Enriched and expanded T cells can be tetramer or dimer sorted to generate a highly pure population of antigen-specific T cells for TCR sequencing. This is a rapid way to identify and generate enough material for sufficient TCR sequencing in a very short time.

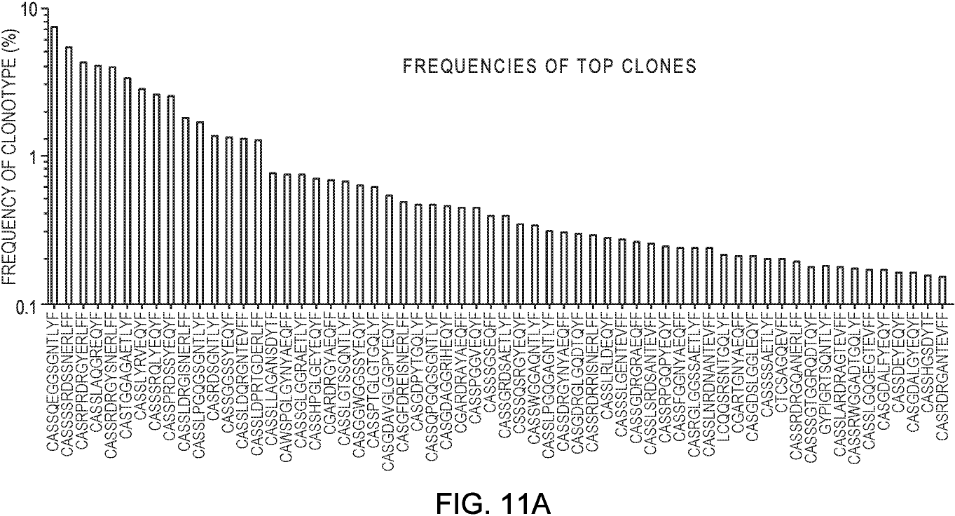

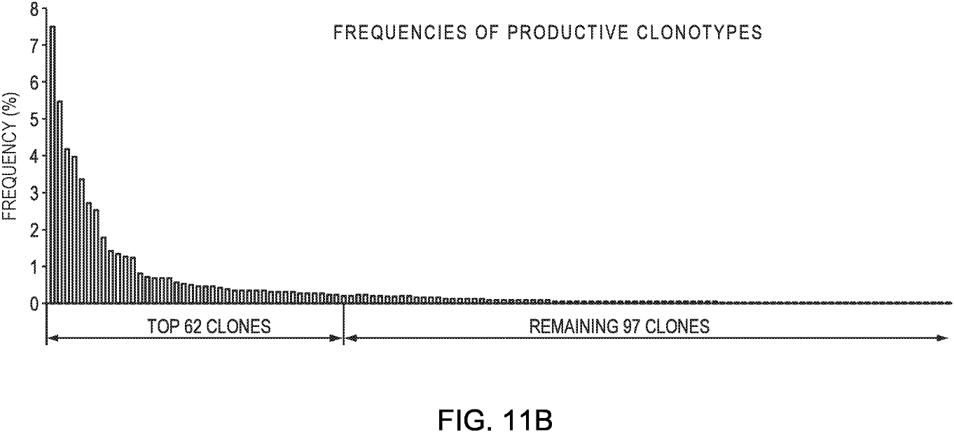

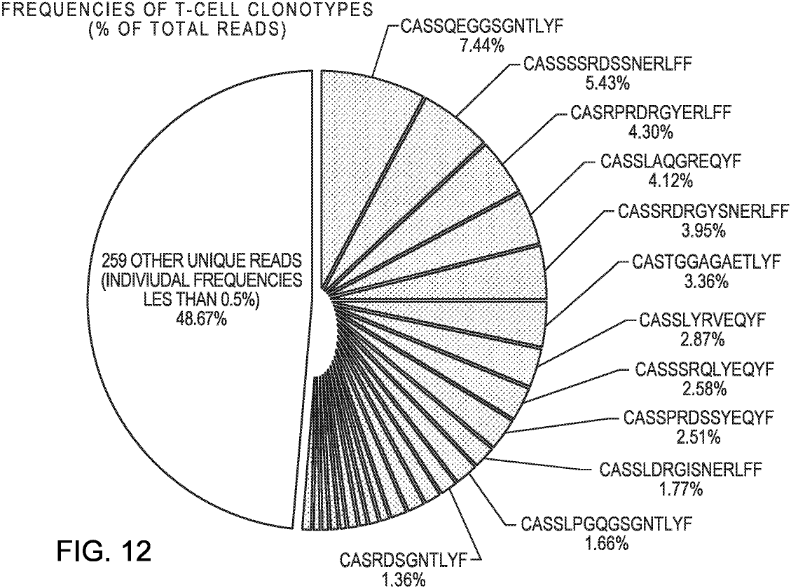

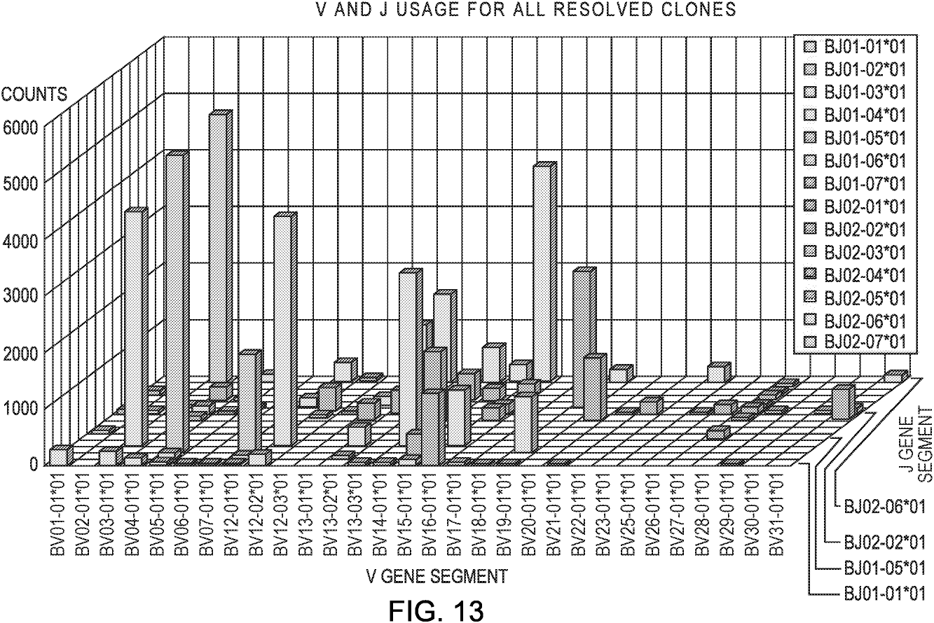

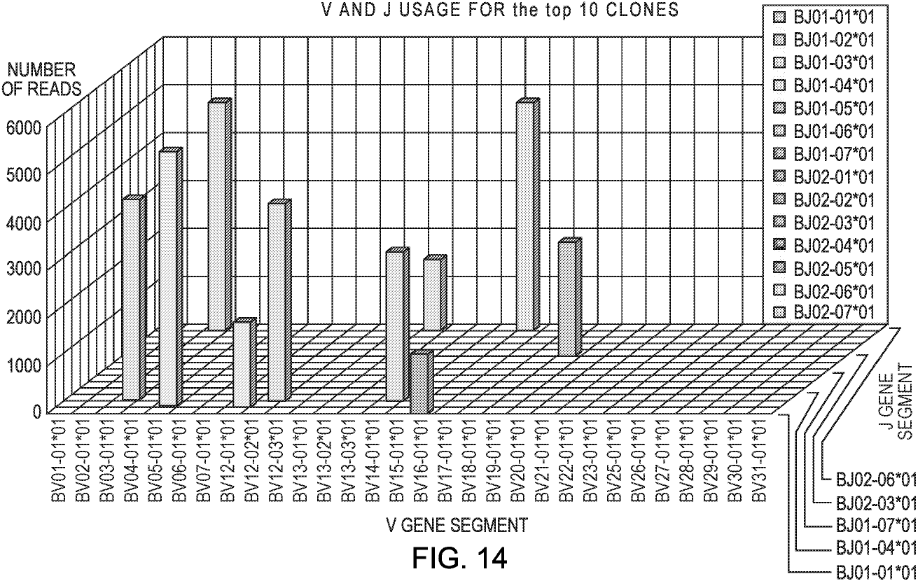

Magnetic enrichment resulted in high frequencies of productive clonotypes. FIG. 11A, FIG. 11B, FIG. 12. These results compare nicely with the results of Carreno et al. (FIG. 11A), where frequencies were more evenly distributed. Clones can be evaluated for V and J pairing frequency (FIG. 13, FIG. 14).

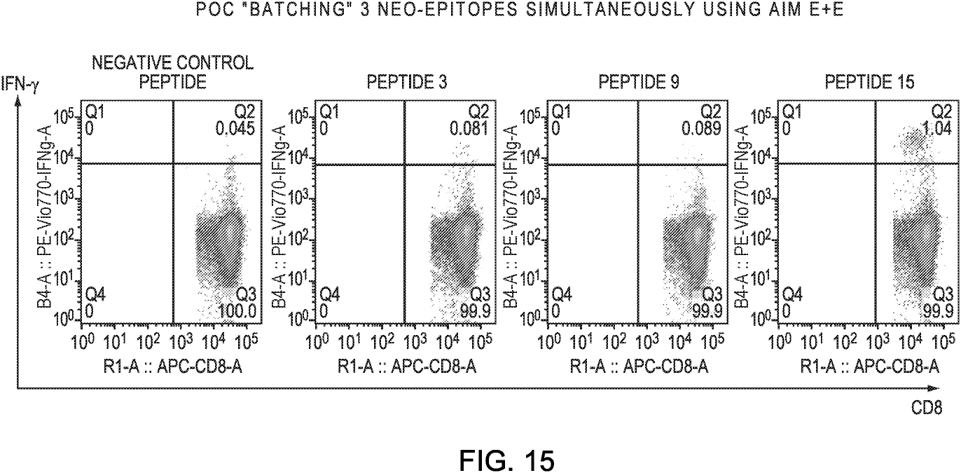

Magnetic enrichment and expansion allows for T cell populations to be screened for reactivity against candidate antigens, including neoantigens. Screening can be conducted in a batched manner. FIG. 15. For example, functionally active human neo-antigen-specific CD-8+ T cells were identified from a healthy donor. Three neo-epitopes from MCF-7 breast cancer were tested simultaneously using the magnetic enrichment and expansion process. Thus, response of a polyclonal CD8 T cell population can be detected against predicted neo-epitopes from mutated antigens. Since these T cell populations are typically very rare it is often not possible to detect them with conventional techniques such as tetramer analysis.

Sequential enrichment makes this process more efficient. With sequential enrichment, the negative cell population of a magnetic enrichment step (that contains only unbound T cells that were negative for the desired antigen) are then incubated with new nanoparticles loaded with another set of antigenic-peptides. This process can be repeated multiple times (e.g., at least 6 times) with 10-15 different peptide loaded nanoparticles in each run. This enables the sequential E+E approach to probe a single sample for a minimum of 90 different antigens.

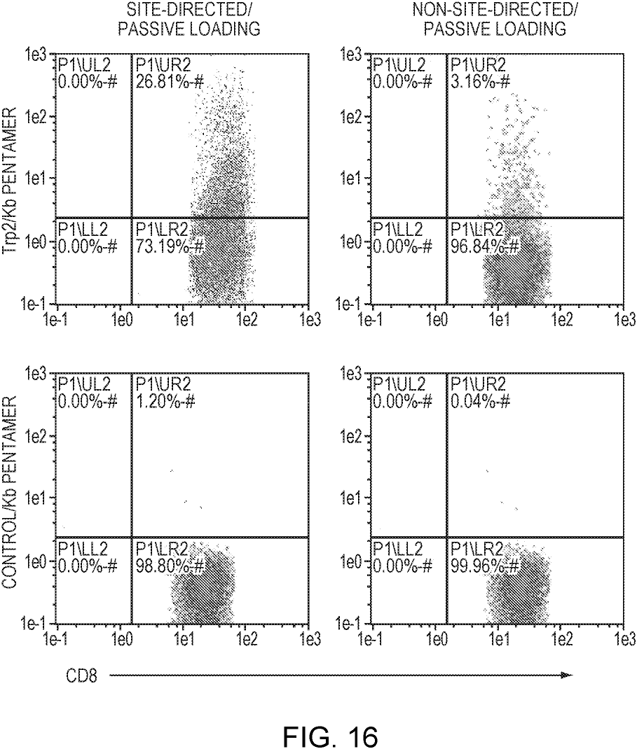

FIG. 16 shows that passive loading of peptide to nanoparticles having site-directed MHC conjugation provided an increased expansion after 1 week. CD8+ T cells were isolated from naïve C57BL/6 spleens and incubated with nanoparticles (Kb Ig dimer/aCD28) loaded with Trp2 peptide at 20 uL particles per 10⁷ cells for 1 hour at 4° C. Then cells bound to the nanoparticles were isolated using a magnetic column and cultured at a 96 well plate for 7 days. At Day 7, cells were harvested, counted and stained with anti-CD8 antibody and Trp2/Kb pentamer. For control, Kb pentamer with irrelevant peptides were used.

The design and construction of nanoparticles in which sig. 1 and sig. 2 are covalently bound in a site directed manner via an engineered free cysteine at the FC end of the molecule makes them very stable with long shelf live. This allows for production of large unloaded batches that are later passively loaded with peptides of interest. For example, during the loading process unloaded particles are incubated with an excess of peptide at 4° C. for a minimum of 3 days. Afterwards the unbound excess of free peptide is removed by washing the loaded nanoparticles on a magnetic column. The paramagnetic particles will be retained on the column and the free peptide will be washed away. After intense washing (3-5 times) the magnet will be removed and the particles are eluted. This passive loading approach introduces high antigenic flexibility to the system, reduces manufacturing cost and enables batching approaches for generation of custom made patient specific multi-antigen/particle cocktails (5-10 antigens), and enabled high throughput screening for neo-epitope identification (>50 epitopes).

Current performance uses an approximately 1:1 ratio for signal 1 and signal 2 (anti-CD28) and high protein density (80-200 ligands) per particle. Particles are in the range of 50 to 150 nm.

What is claimed is:

1. A method for identifying an antigen-specific T cell Receptor (TCR), comprising:
magnetically enriching and expanding a heterogeneous T cell population with paramagnetic nanoparticles having an MHC-peptide antigen presenting complex on the surface of the nanoparticles,
sorting the expanded T cells with the MHC-peptide ligand, to obtain a T cell population with desired antigen specificity; and
sequencing the TCR genes or portions thereof in the T cell population.

2-10. (canceled)

11. A method for screening a T cell population for reactivity to a library of antigenic peptides, comprising:
magnetically enriching and expanding antigen-specific T cells in the population with a cocktail of paramagnetic nanoparticles, each having a surface-conjugated MHC-peptide antigen presenting complex that presents an antigenic peptide of interest,
and phenotypically evaluating the expanded T cells.

12-23. (canceled)

24. A method for expansion of T cells comprising a heterologous or engineered T cell receptor (TCR), comprising:
magnetically enriching and expanding a T cell population comprising T cells expressing a heterologous or engineered T cell receptor (TCR), with paramagnetic nanoparticles having an MHC-peptide antigen presenting complex on the surface thereof that is recognized by the heterologous or engineered T cell receptor (TCR).

25-27. (canceled)

28. A method for preparing an antigen-specific T-cell population, comprising:
providing a sample comprising T cells from a patient or a suitable donor;
contacting said sample with first nanoparticles which are paramagnetic and comprise on their surface an MHC-peptide antigen-presenting complex, wherein the MHC-peptide complex is prepared by passive loading of MHC-conjugated nanoparticles;
placing a magnetic field in proximity to the paramagnetic nanoparticles,
recovering antigen-specific T cells associated with the paramagnetic particles, and
optionally expanding the recovered T cells in the presence of a magnetic field.

29-45. (canceled)

46. A method for generating a T cell expressing a chimeric antigen receptor (CAR), comprising:
magnetically enriching and expanding a T cell population with paramagnetic nanoparticles having an MHC-peptide antigen presenting complex on the surface thereof, to thereby prepare an enriched and expanded antigen-specific T cell population; and
transforming the T cell population with a chimeric antigen receptor (CAR).

47-63. (canceled)

64. A method for expanding a T cell expressing a CAR, comprising:
providing the T cell population expressing a CAR according to claim 46, and
magnetically expanding the T cell population in the presence of paramagnetic nanoparticles having an MHC-peptide antigen presenting complex on the surface thereof.

65. (canceled)

66. A method for treating a patient having cancer, comprising:
administering the CAR-T prepared according to the method of claim 46, and
administering an artificial antigen presenting cell to the patient, presenting the antigen of interest in complex with MHC, and a lymphocyte costimulatory ligand.

67. A method for treating a patient having hematological cancer that has relapsed after allogeneic stem cell transplantation, comprising:
providing a sample comprising T cells from a suitable donor;
contacting said sample with nanoparticles which are paramagnetic and comprise on their surface: (1) an MHC-peptide antigen-presenting complex, wherein the MHC-peptide complex is prepared by passive loading of MHC-conjugated nanoparticles (signal 1); and (2) an anti-CD28 co-stimulatory ligand (signal 2);
placing a magnetic field in proximity to the paramagnetic nanoparticles,
recovering antigen-specific T cells associated with the paramagnetic particles,
expanding the recovered T cells; and
administering expanded T cells to the patient.

68-76. (canceled)

77. A method for preparing a cytotoxic T lymphocyte (CTL) population comprising at least 10⁶ CTLs having a central memory or effector memory phenotype, the method comprising:
providing CD8+ T cells isolated from a peripheral blood mononuclear cell (PBMC) sample from a patient or donor, the CD8+ cells being isolated by positive or negative selection,
contacting said sample with paramagnetic nanoparticles having a size in the range of about 10 to about 250 nm, which comprise on their surfaces MHC Class I-peptide antigen-presenting complexes and lymphocyte co-stimulatory ligands, wherein the MHC Class I-peptide antigen presenting complex ligands and the lymphocyte co-stimulatory ligands are present on the same of different nanoparticles;
placing a magnetic field in proximity to the paramagnetic nanoparticles for about 5 minutes,
separating a magnetic fraction from a non-magnetic fraction, and
expanding the magnetic fraction in culture for 2 to 3 weeks to prepare the CTL population.

78. The method of claim 77, wherein the MHC Class I-peptide antigen-presenting complex ligands present at least two tumor associated antigens.

79. The method of claim 78, wherein the tumor-associated antigens are AML-associated antigens.

80. The method of claim 79, wherein the AML-associated antigens include one or more selected from: Survivin, WT-1, PRAME, RHAMM, and PR3.

81. The method of claim 77, wherein the lymphocyte co-stimulatory ligands are B7.1 or an activating antibody against CD28.

82. The method of claim 77, wherein the MHC Class I-peptide antigen presenting complexes are HLA-Ig ligands.

83. The method of claim 77, wherein the CTL population comprises at least 10⁸ CTLs.

84. The method of claim 77, wherein the sample comprises at least about 10⁶ CD8-enriched cells.

85. The method of claim 77, wherein the paramagnetic nanoparticles are dextran-coated iron oxide nanoparticles.

86. The method of claim 77, wherein the CTL population is at least 10% specific for the peptide antigen(s).
